(19) United States
(12) Patent Application Publication
(10) Pub. No.: US 2023/0201192 A1
(43) Pub. Date: Jun. 29, 2023

(54) METHODS AND MATERIALS FOR ASSESSING AND TREATING CANCER

(71) Applicant: Mayo Foundation for Medical Education and Research, Rochester, MN (US)

(72) Inventors: John A. Copland, III, Ponte Vedra Beach, FL (US); Laura Ann Marlow, Jacksonville, FL (US)

(21) Appl. No.: 17/928,503

(22) PCT Filed: Jun. 16, 2021
(86) PCT No.: PCT/US2021/037625

§ 371 Date: Nov. 29, 2022

Related U.S. Application Data
(60) Provisional application No. 63/041,570, filed on Jun. 19, 2020.

(51) Int. Cl. A61K 31/496; A61K 45/06; A61P 35/00; C12Q 1/6886

(52) U.S. Cl. CPC A61K 31/496; A61K 45/06; A61P 35/00; C12Q 1/6886; C12Q 2600/106; C12Q 2600/158; G01N 2800/52

(57) ABSTRACT

This document relates to methods and materials involved in assessing and/or treating mammals (e.g., humans) having cancer (e.g., a SCD1-associated cancer). For example, methods for determining whether or not a cancer is likely to be responsive to one or more stearoyl CoA desaturase 1 (SCD1) polypeptide inhibitors (e.g., a selective SCD1 inhibitor (SSI)) are provided. In some cases, the methods and materials for treating a mammal by administering, to the mammal, one or more cancer treatments that is/are selected based, at least in part, on whether or not the mammal is likely to be responsive to one or more SCD1 polypeptide inhibitors e.g., SSI-4) are provided.

### CROSS-REFERENCE TO RELATED APPLICATIONS

This application claims the benefit of U.S. Patent Application Ser. No. 63/041,570, filed on Jun. 19, 2020. The disclosure of the prior application is considered part of (and is incorporated by reference in) the disclosure of this application.

### STATEMENT REGARDING FEDERAL FUNDING

This invention was made with government support under CA210964 and CA015083 awarded by the National Institutes of Health. The government has certain rights in the invention.

### TECHNICAL FIELD

This document relates to methods and materials involved in assessing and/or treating mammals (e.g., humans) having cancer (e.g., a SCD1-associated cancer). For example, methods and materials provided herein can be used to determine whether or not a cancer is likely to be responsive to one or more stearoyl CoA desaturase 1 (SCD1) polypeptide inhibitors (e.g., a selective SCD1 inhibitor (SSI)). In some cases, the methods and materials provided herein can be used to treat a mammal by administering, to the mammal, one or more cancer treatments that is/are selected based, at least in part, on whether or not the mammal is likely to be responsive to one or more SCD1 polypeptide inhibitors (e.g., SSI-4).

### BACKGROUND INFORMATION

While new therapies have been FDA approved for many cancers, including hepatocellular carcinoma (HCC), renal cell carcinoma (RCC), cholangiocarcinoma (CCA) and melanoma (MM), there remains a need for novel effective therapeutic agents. Recent studies indicate lipid biosynthesis and desaturation is required for HCC survival. Stearoyl CoA desaturase (SCD1), a key mediator of fatty acid (FA) biosynthesis and rate-limiting in conversion of saturated fatty acids (SFAs) to mono-unsaturated fatty acids (MUFAs), is upregulated in many cancers.

### SUMMARY

SCD1 is an enzyme that catalyzes the de novo lipogenesis of Δ-9 monounsaturated fatty acids (MUFA) oleic acid (OA) and palmitoleic acid (PA). These MUFAs are essential for the synthesis of triglycerides, sphingolipids, ceramides, glycolipids, phospholipids, and other lipoproteins which influence membrane fluidity, membrane raft formation and receptor clustering, second messenger signaling, fatty acid oxidation, energy storage, cell division, inflammation, and a number of other biological functions (Guillou et al., 2010 Prog Lipid Res. 49(2):186-199). Aberrant upregulation of SCD1 has been implicated in the development of certain types of cancer; however, not all SCD1-associated cancers are responsive to SCD1 polypeptide inhibitors. As used herein, a cancer associated with expression (e.g., overexpression) of a SCD1 polypeptide can also be referred to as a SCD1-associated cancer (see, e.g., von Roemeling et al. 2015 J. Clin. Endocrinol. Metab. 100:E697-E709).

This document provides methods and materials involved in assessing and/or treating mammals (e.g., humans) having cancer (e.g., a SCD1-associated cancer). In some cases, this document provides methods and materials for determining whether or not a mammal having cancer is likely to be responsive to one or more SCD1 polypeptide inhibitors (e.g., SSI-4), and, optionally, administering one or more cancer treatments that is/are selected based, at least in part, on whether or not the mammal is likely to be responsive to one or more SCD1 polypeptide inhibitors to the mammal. For example, a sample (e.g., a sample containing one or more cancer cells) obtained from a mammal (e.g., a human) having cancer can be assessed to determine if the mammal is likely to be responsive to one or more SCD1 polypeptide inhibitors (e.g., SSI-4) based, at least in part, on the presence, absence, or level of phosphorylated Src (p-Src) polypeptide expression in the sample.

As demonstrated herein, the level of expression of polypeptides that promote glycolysis in a cancer cell can be correlated with SCDI inhibitor responsiveness. For example, the presence of a reduced level of p-Src polypeptide expression in a SCD1-associated cancer can indicate that the SCD1-associated cancer is likely to be responsive to treatment with one or more SCD1 polypeptide inhibitors (e.g., SSI-4). For example, the presence of an elevated level of c-Myc polypeptide expression in a cancer cell and/or the presence of an elevated level of lactate dehydrogenase A (LDHA) polypeptide expression in a cancer cell can indicate that the SCD1-associated cancer is likely to be responsive to treatment with one or more SCD1 polypeptide inhibitors (e.g., SSI-4).

Having the ability to determine whether or not a particular patient is likely to respond to one or more SCD1 polypeptide inhibitors (e.g., SSI-4) allows clinicians to provide an individualized approach in selecting cancer treatments for patients.

In general, one aspect of this document features methods for determining whether or not a mammal having a SCD1-associated cancer is likely to respond to treatment with a SSI. The methods can include, or consist essentially of, (a) detecting a presence or absence of a decreased level of p-Src polypeptide expression in a sample from a mammal; and (b) identifying the mammal as being likely to respond to the SSI if the presence of the decreased level is detected, or (c) identifying the mammal as not being likely to respond to the SSI if the absence of the decreased level is detected. The mammal can be a human. The sample can include cancer cells of the cancer. The cancer can be a solid tumor, and can be a liver cancer, a renal cell carcinoma, an ovarian cancer, a breast cancer, a prostate cancer, a colon cancer, a pancreatic cancer, a bladder cancer, a lung cancer, a thyroid cancer, a melanoma, a brain cancer, a stomach cancer, a cervical cancer, a uterine cancer, a chronic lymphocytic leukemia, a, acute lymphocytic leukemia, or a lymphoma. The cancer can be a liver cancer (e.g., a hepatocellular carcinoma or a cholangiocarcinoma). The method can include detecting a presence or absence of an elevated level of c-Myc polypeptide expression in a sample from the mammal. The method can include detecting a presence or absence of an elevated level of LDHA polypeptide expression in a sample from the mammal. The method can include detecting the presence of the decreased level of p-Src polypeptide expression, detecting the presence of the elevated level of c-Myc polypeptide expression, and detecting the presence of the elevated level of LDHA polypeptide expression, and can include identifying the mammal as being likely to respond to the SSI. Detecting the presence of the decreased level of p-Src polypeptide expression, detecting the presence of the elevated level of c-Myc polypeptide expression, and detecting the presence of the elevated level of LDHA polypeptide expression can include immunohistochemistry. The decreased level of p-Src polypeptide expression can include an IHC intensity level of 0 or 1, the elevated level of c-Myc polypeptide expression can include nuclear c-Myc polypeptide expression in greater than about 40% of cells in the sample, and the elevated level of LDHA polypeptide expression can include an IHC intensity level of 3 or 4. The method can include detecting the absence of the decreased level of p-Src polypeptide expression, detecting the absence of the elevated level of c-Myc polypeptide expression, and detecting the absence of the elevated level of LDHA polypeptide expression, and method can include identifying the mammal as not being likely to respond to the SSI. The SCD1 polypeptide inhibitor can be a compound having Formula (II) or Formula (IIa):

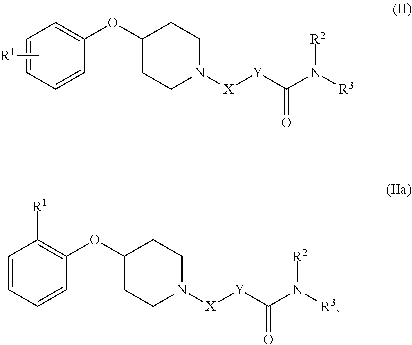

or a pharmaceutically acceptable salt thereof; where R¹ is halo; X is —(C═O)NR⁴—; Y is

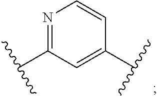 [SMILES: *c1ccnc(*)c1]

and R², R³, and R⁴ are each independently H or an unsubstituted C₁₋₆alkyl. The SCD1 polypeptide inhibitor can be SSI-4, 2-{[4-(2-Chlorophenoxy)piperidine-1-carbonyl]amino}-N-methylpyridine-4-carboxamide or a pharmaceutically acceptable salt thereof. The SCD1 polypeptide inhibitor can be a compound having Formula (I) or Formula (Ia):

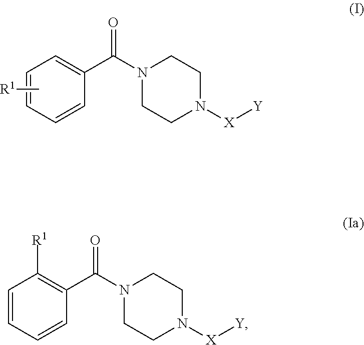

or a pharmaceutically acceptable salt thereof; where R¹ is an unsubstituted C₁₋₆alkyl or C₁₋₆ haloalkyl; X is

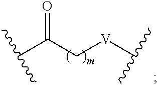

Y is selected from:

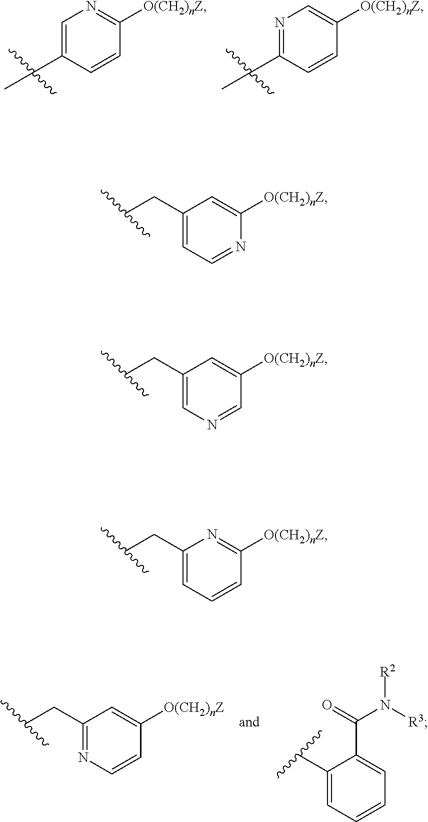

m is 0 or 1; n is 0, 1, or 2; V is NR⁴ or O; R², R³, and R⁴ are each independently H or an unsubstituted C₁₋₆alkyl; and Z is an unsubstituted aryl. The SCD1 polypeptide inhibitor can be SSI-2,2-(benzyloxy)-5-{[hydroxy({4-[2-(trifluoromethyl)benzoyl]piperazin-1-yl})methyl]amino}-1,2-dihydropyridin-2-ylium-1-ide, or a pharmaceutically acceptable salt thereof.

In another aspect, this document features methods for treating a mammal having a SCD1-associated cancer. The methods can include, or consist essentially of, (a) detecting a decreased level of p-Src polypeptide expression in a sample obtained from a mammal; and (b) administering a SCD1 polypeptide inhibitor to the mammal. The mammal can be a human. The sample can include cancer cells of the cancer. The cancer can include a solid tumor, and can be a liver cancer, a renal cell carcinoma, an ovarian cancer, a breast cancer, a prostate cancer, a colon cancer, a pancreatic cancer, a bladder cancer, a lung cancer, a thyroid cancer, a melanoma, a brain cancer, a stomach cancer, a cervical cancer, a uterine cancer, a chronic lymphocytic leukemia, a, acute lymphocytic leukemia, or a lymphoma. The cancer can be a liver cancer (e.g., a hepatocellular carcinoma or a cholangiocarcinoma). The method can include detecting an elevated level of c-Myc polypeptide expression in a sample from the mammal. The method can include detecting an elevated level of LDHA polypeptide expression in a sample from the mammal. The method can include detecting the presence of the decreased level of p-Src polypeptide expression, detecting the presence of the elevated level of c-Myc polypeptide expression, and detecting the presence of the elevated level of LDHA polypeptide expression. Detecting the presence of the decreased level of p-Src polypeptide expression, detecting the presence of the elevated level of c-Myc polypeptide expression, and detecting the presence of the elevated level of LDHA polypeptide expression can include immunohistochemistry. The decreased level of p-Src polypeptide expression can include an IHC intensity level of 0 or 1, the elevated level of c-Myc polypeptide expression can include nuclear c-Myc polypeptide expression in greater than about 40% of cells in said sample, and the elevated level of LDHA polypeptide expression can include an IHC intensity level of 3 or 4. The SCD1 polypeptide inhibitor can be a compound having Formula (II) or Formula (IIa), or a pharmaceutically acceptable salt thereof; where R¹ is halo; X is —(C═O)NR⁴—; Y is

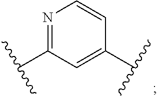 [SMILES: *c1ccnc(*)c1]

and R², R³, and R⁴ are each independently H or an unsubstituted C₁₋₆alkyl. The SCD1 polypeptide inhibitor can be SSI-4, 2-{[4-(2-Chlorophenoxy)piperidine-1-carbonyl]amino}-N-methylpyridine-4-carboxamide, or a pharmaceutically acceptable salt thereof. The SCD1 polypeptide inhibitor can be a compound having Formula (I) or Formula (Ia), or a pharmaceutically acceptable salt thereof; where R¹ is an unsubstituted C₁₋₆alkyl or C₁₋₆haloalkyl; X is

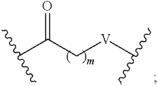

Y is selected from:

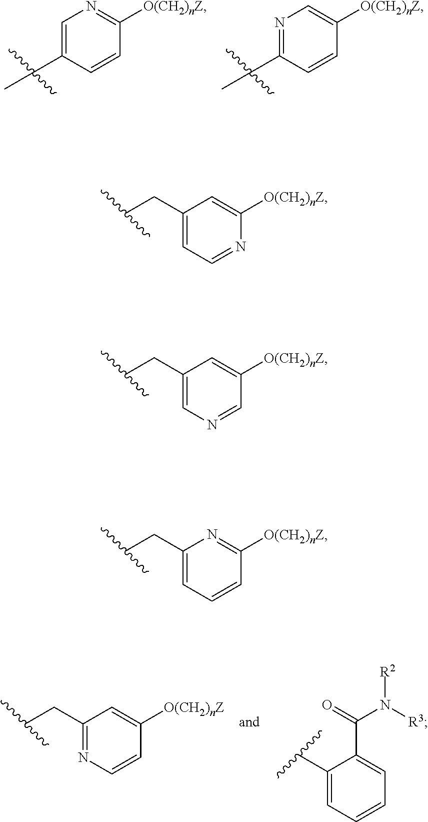

m is 0 or 1; n is 0, 1, or 2; V is NR⁴ or O; R², R³, and R⁴ are each independently H or an unsubstituted C₁₋₆alkyl; and Z is an unsubstituted aryl. The SCD1 polypeptide inhibitor can be SSI-2,2-(benzyloxy)-5-{[hydroxy({4-[2-(trifluoromethyl)benzoyl]piperazin-1-yl})methyl]amino}-1,2-dihydropyridin-2-ylium-1-ide, or a pharmaceutically acceptable salt thereof. The method can include administering a cancer treatment to the mammal. The cancer treatment can include a kinase inhibitor (e.g., regorafenib). The cancer treatment can include a mTOR inhibitor. The cancer treatment can include a proteosome inhibitor. The cancer treatment can include an immune checkpoint inhibitor.

In another aspect, this document features methods for treating a SCD1-associated cancer. The methods can include, or consist essentially of, administering a SCD1 polypeptide inhibitor to a mammal identified as having a decreased level of p-Src polypeptide expression in a sample obtained from the mammal. The mammal can be a human. The sample can include cancer cells of the cancer. The cancer can include a solid tumor, and can be a liver cancer, a renal cell carcinoma, an ovarian cancer, a breast cancer, a prostate cancer, a colon cancer, a pancreatic cancer, a bladder cancer, a lung cancer, a thyroid cancer, a melanoma, a brain cancer, a stomach cancer, a cervical cancer, a uterine cancer, a chronic lymphocytic leukemia, a, acute lymphocytic leukemia, or a lymphoma. The cancer can be a liver cancer (e.g., a hepatocellular carcinoma or a cholangiocarcinoma). The method can include detecting an elevated level of c-Myc polypeptide expression in a sample from the mammal. The method can include detecting an elevated level of LDHA polypeptide expression in a sample from the mammal. The method can include detecting the presence of the decreased level of p-Src polypeptide expression, detecting the presence of the elevated level of c-Myc polypeptide expression, and detecting the presence of the elevated level of LDHA polypeptide expression. Detecting the presence of the decreased level of p-Src polypeptide expression, detecting the presence of the elevated level of c-Myc polypeptide expression, and detecting the presence of the elevated level of LDHA polypeptide expression can include immunohistochemistry. The decreased level of p-Src polypeptide expression can include an IHC intensity level of 0 or 1, the elevated level of c-Myc polypeptide expression can include nuclear c-Myc polypeptide expression in greater than about 40% of cells in said sample, and the elevated level of LDHA polypeptide expression can include an IHC intensity level of 3 or 4. The SCD1 polypeptide inhibitor can be a compound having Formula (II) or Formula (IIa), or a pharmaceutically acceptable salt thereof; where R¹ is halo; X is —(C═O)NR⁴—; Y is

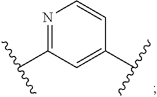 [SMILES: *c1ccnc(*)c1]

and R², R³, and R⁴ are each independently H or an unsubstituted C₁₋₆alkyl. The SCD1 polypeptide inhibitor can be SSI-4, 2-{[4-(2-Chlorophenoxy)piperidine-1-carbonyl]amino}-N-methylpyridine-4-carboxamide, or a pharmaceutically acceptable salt thereof. The SCD1 polypeptide inhibitor can be a compound having Formula (I) or Formula (Ia), or a pharmaceutically acceptable salt thereof where R¹ is an unsubstituted C₁₋₆alkyl or C₁₋₆haloalkyl; X is

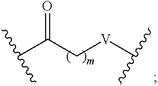

Y is selected from:

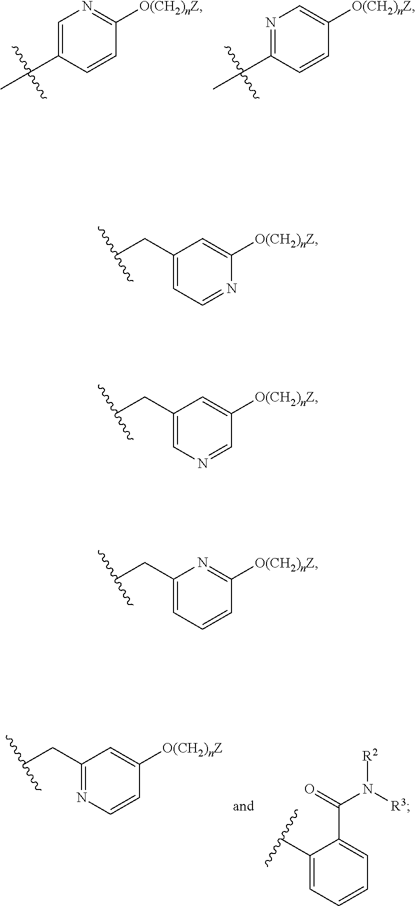

m is 0 or 1; n is 0, 1, or 2; V is NR⁴ or O; R², R³, and R⁴ are each independently H or an unsubstituted C₁₋₆alkyl; and Z is an unsubstituted aryl. The SCD1 polypeptide inhibitor can be SSI-2,2-(benzyloxy)-5-{[hydroxy({4-[2-(trifluoromethyl)benzoyl]piperazin-1-yl})methyl]amino}-1,2-dihydropyridin-2-ylium-1-ide, or a pharmaceutically acceptable salt thereof. The method can include administering a cancer treatment to the mammal. The cancer treatment can include a kinase inhibitor (e.g., regorafenib). The cancer treatment can include a mTOR inhibitor. The cancer treatment can include a proteosome inhibitor. The cancer treatment can include an immune checkpoint inhibitor.

In another aspect, this document features methods for treating a mammal having a SCD1-associated cancer. The methods can include, or consist essentially of, (a) detecting an absence of a decreased level of p-Src polypeptide expression in a sample obtained from a mammal; and (b) administering a cancer treatment to the mammal, where the cancer treatment is not a SCD1 polypeptide inhibitor. The mammal can be a human. The sample can include cancer cells of the cancer. The cancer can include a solid tumor, and can be a liver cancer, a renal cell carcinoma, an ovarian cancer, a breast cancer, a prostate cancer, a colon cancer, a pancreatic cancer, a bladder cancer, a lung cancer, a thyroid cancer, a melanoma, a brain cancer, a stomach cancer, a cervical cancer, a uterine cancer, a chronic lymphocytic leukemia, a, acute lymphocytic leukemia, or a lymphoma. The cancer can be a liver cancer (e.g., a hepatocellular carcinoma or a cholangiocarcinoma). The method can include detecting an absence of an elevated level of c-Myc polypeptide expression in a sample from the mammal. The method can include detecting an absence of an elevated level of LDHA polypeptide expression in a sample from the mammal. The method can include detecting the absence of the decreased level of p-Src polypeptide expression, detecting the absence of the elevated level of c-Myc polypeptide expression, and detecting the absence of the elevated level of LDHA polypeptide expression. The cancer treatment can be surgery. The cancer treatment can be radiation therapy.

In another aspect, this document features methods for treating a SCD1-associated cancer. The methods can include, or consist essentially of, administering a SCD1 polypeptide inhibitor to a mammal identified as having an absence of a decreased level of p-Src polypeptide expression in a sample obtained from a mammal. The mammal can be a human. The sample can include cancer cells of the cancer. The cancer can include a solid tumor, and can be a liver cancer, a renal cell carcinoma, an ovarian cancer, a breast cancer, a prostate cancer, a colon cancer, a pancreatic cancer, a bladder cancer, a lung cancer, a thyroid cancer, a melanoma, a brain cancer, a stomach cancer, a cervical cancer, a uterine cancer, a chronic lymphocytic leukemia, a, acute lymphocytic leukemia, or a lymphoma. The cancer can be a liver cancer (e.g., a hepatocellular carcinoma or a cholangiocarcinoma). The method can include detecting an absence of an elevated level of c-Myc polypeptide expression in a sample from the mammal. The method can include detecting an absence of an elevated level of LDHA polypeptide expression in a sample from the mammal. The method can include detecting the absence of the decreased level of p-Src polypeptide expression, detecting the absence of the elevated level of c-Myc polypeptide expression, and detecting the absence of the elevated level of LDHA polypeptide expression. The cancer treatment can be surgery. The cancer treatment can be radiation therapy.

In another aspect, this document features methods for determining whether or not a mammal having a SCD1-associated cancer is likely to respond to treatment with a SSI. The methods can include, or consist essentially of, (a) plating a cell from a sample from a mammal on soft agar; and (b) identifying the mammal as being likely to respond to the SSI if the cell forms a colony in the soft agar, or (c) identifying the mammal as not being likely to respond to the SSI if said cell does not form a colony in the soft agar. The mammal can be a human. The sample can include cancer cells of the cancer. The cancer can include a solid tumor, and can be a liver cancer, a renal cell carcinoma, an ovarian cancer, a breast cancer, a prostate cancer, a colon cancer, a pancreatic cancer, a bladder cancer, a lung cancer, a thyroid cancer, a melanoma, a brain cancer, a stomach cancer, a cervical cancer, a uterine cancer, a chronic lymphocytic leukemia, a, acute lymphocytic leukemia, or a lymphoma. The cancer can be a liver cancer (e.g., a hepatocellular carcinoma or a cholangiocarcinoma).

In another aspect, this document features methods for treating a mammal having a SCD1-associated cancer. The methods can include, or consist essentially of, (a) detecting soft agar colony formation from a single cell from a sample obtained from a mammal; and (b) administering a SCD1 polypeptide inhibitor to the mammal. The mammal can be a human. The sample can include cancer cells of the cancer. The cancer can include a solid tumor, and can be a liver cancer, a renal cell carcinoma, an ovarian cancer, a breast cancer, a prostate cancer, a colon cancer, a pancreatic cancer, a bladder cancer, a lung cancer, a thyroid cancer, a melanoma, a brain cancer, a stomach cancer, a cervical cancer, a uterine cancer, a chronic lymphocytic leukemia, a, acute lymphocytic leukemia, or a lymphoma. The cancer can be a liver cancer (e.g., a hepatocellular carcinoma or a cholangiocarcinoma). The SCD1 polypeptide inhibitor can be a compound having Formula (II) or Formula (IIa), or a pharmaceutically acceptable salt thereof; where R¹ is halo; X is —(C═O)NR⁴—; Y is

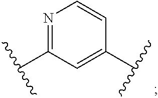 [SMILES: *c1ccnc(*)c1]

and R², R³, and R⁴ are each independently H or an unsubstituted C₁₋₆alkyl. The SCD1 polypeptide inhibitor can be SSI-4, 2-{[4-(2-Chlorophenoxy)piperidine-1-carbonyl]amino}-N-methylpyridine-4-carboxamide, or a pharmaceutically acceptable salt thereof. The SCD1 polypeptide inhibitor can be a compound having Formula (I) or Formula (Ia), or a pharmaceutically acceptable salt thereof; where R¹ is an unsubstituted C₁₋₆alkyl or C₁₋₆haloalkyl; X is

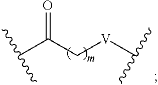

Y is selected from:

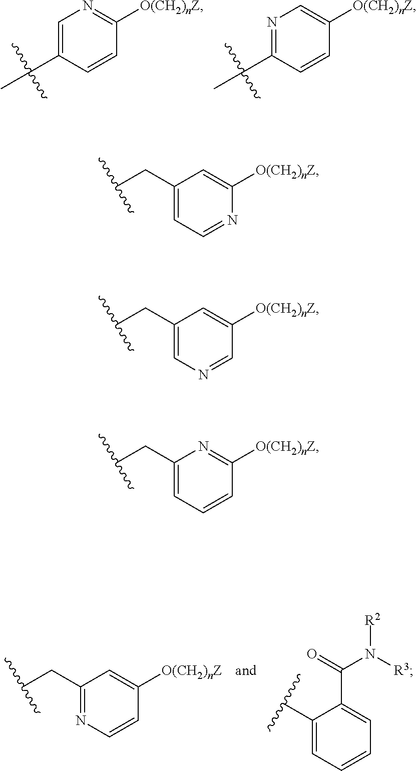

m is 0 or 1; n is 0, 1, or 2; V is NR⁴ or O; R², R³, and R⁴ are each independently H or an unsubstituted C₁₋₆alkyl; and Z is an unsubstituted aryl. The SCD1 polypeptide inhibitor can be SSI-2,2-(benzyloxy)-5-{[hydroxy({4-[2-(trifluoromethyl)benzoyl]piperazin-1-yl})methyl]amino}-1,2-dihydropyridin-2-ylium-1-ide, or a pharmaceutically acceptable salt thereof. The method can include administering a cancer treatment to the mammal. The cancer treatment can include a kinase inhibitor (e.g., regorafenib). The cancer treatment can include a mTOR inhibitor. The cancer treatment can include a proteosome inhibitor. The cancer treatment can include an immune checkpoint inhibitor.

In another aspect, this document features methods for treating a SCD1-associated cancer. The methods can include, or consist essentially of, administering a SCD1 polypeptide inhibitor to a mammal identified as having soft agar colony formation from a single cell from a sample obtained from the mammal. The mammal can be a human. The sample can include cancer cells of the cancer. The cancer can include a solid tumor, and can be a liver cancer, a renal cell carcinoma, an ovarian cancer, a breast cancer, a prostate cancer, a colon cancer, a pancreatic cancer, a bladder cancer, a lung cancer, a thyroid cancer, a melanoma, a brain cancer, a stomach cancer, a cervical cancer, a uterine cancer, a chronic lymphocytic leukemia, a, acute lymphocytic leukemia, or a lymphoma. The cancer can be a liver cancer (e.g., a hepatocellular carcinoma or a cholangiocarcinoma). The SCD1 polypeptide inhibitor can be a compound having Formula (II) or Formula (IIa), or a pharmaceutically acceptable salt thereof; where R¹ is halo; X is —(C═O)NR⁴—; Y is

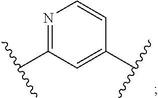 [SMILES: *c1ccnc(*)c1]

and R², R³, and R⁴ are each independently H or an unsubstituted C₁₋₆alkyl. The SCD1 polypeptide inhibitor can be SSI-4, 2-{[4-(2-Chlorophenoxy)piperidine-1-carbonyl]amino}-N-methylpyridine-4-carboxamide, or a pharmaceutically acceptable salt thereof. The SCD1 polypeptide inhibitor can be a compound having Formula (I) or Formula (Ia), or a pharmaceutically acceptable salt thereof; where R¹ is an unsubstituted C₁₋₆alkyl or C₁₋₆haloalkyl; X is

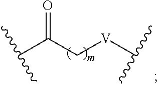

Y is selected from:

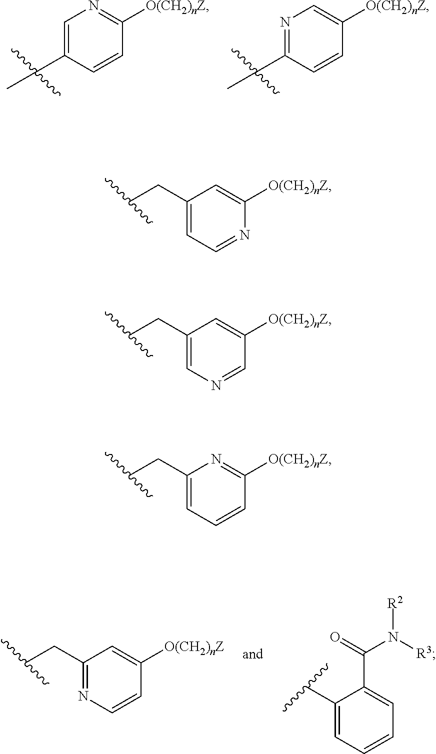

m is 0 or 1; n is 0, 1, or 2; V is NR⁴ or O; R², R³, and R⁴ are each independently H or an unsubstituted C₁₋₆alkyl; and Z is an unsubstituted aryl. The SCD1 polypeptide inhibitor can be SSI-2,2-(benzyloxy)-5-{[hydroxy({4-[2-(trifluoromethyl)benzoyl]piperazin-1-yl})methyl]amino}-1,2-dihydropyridin-2-ylium-1-ide, or a pharmaceutically acceptable salt thereof. The method can include administering a cancer treatment to the mammal. The cancer treatment can include a kinase inhibitor (e.g., regorafenib). The cancer treatment can include a mTOR inhibitor. The cancer treatment can include a proteosome inhibitor. The cancer treatment can include an immune checkpoint inhibitor.

In another aspect, this document features methods for treating a mammal having a SCD1-associated cancer. The methods can include, or consist essentially of, (a) detecting an absence of soft agar colony formation from a cell from a sample obtained from a mammal; and (b) administering a cancer treatment to the mammal, where the cancer treatment is not a SCD1 polypeptide inhibitor. The mammal can be a human. The sample can include cancer cells of the cancer. The cancer can include a solid tumor, and can be a liver cancer, a renal cell carcinoma, an ovarian cancer, a breast cancer, a prostate cancer, a colon cancer, a pancreatic cancer, a bladder cancer, a lung cancer, a thyroid cancer, a melanoma, a brain cancer, a stomach cancer, a cervical cancer, a uterine cancer, a chronic lymphocytic leukemia, a, acute lymphocytic leukemia, or a lymphoma. The cancer can be a liver cancer (e.g., a hepatocellular carcinoma or a cholangiocarcinoma). The cancer treatment can be surgery. The cancer treatment can be radiation therapy.

In another aspect, this document features methods for treating a SCD1-associated cancer. The methods can include, or consist essentially of, administering a SCD1 polypeptide inhibitor to a mammal identified as having an absence of soft agar colony formation from a cell from a sample obtained from the mammal. The mammal can be a human. The sample can include cancer cells of the cancer. The cancer can include a solid tumor, and can be a liver cancer, a renal cell carcinoma, an ovarian cancer, a breast cancer, a prostate cancer, a colon cancer, a pancreatic cancer, a bladder cancer, a lung cancer, a thyroid cancer, a melanoma, a brain cancer, a stomach cancer, a cervical cancer, a uterine cancer, a chronic lymphocytic leukemia, a, acute lymphocytic leukemia, or a lymphoma. The cancer can be a liver cancer (e.g., a hepatocellular carcinoma or a cholangiocarcinoma). The cancer treatment can be surgery. The cancer treatment can be radiation therapy.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIGS. 1A-1B show viability and SCD1 polypeptide expression in SSI-4 sensitive and insensitive cell lines. FIG. 1A. Viability assay using 1 μM SSI-4 treating cells for 4 days identify SSI-4 sensitive and insensitive cell lines. FIG. 1B. SCD1 protein expression in cell lines demonstrate SSI-4 sensitive and insensitive cell lines.

FIGS. 2A-2D show that SSI-4 has high affinity and specificity for certain tumor cell lines. Dose response of 0.01-10,000 nM SSI-4 with 3 day exposure demonstrates IC50 of 1-40 nM in hepatocellular carcinoma (HCC) responsive cell lines (FIG. 2A), cholangiocarcinoma (CCA) (FIG. 2B), clear cell renal cell carcinoma (ccRCC) (FIG. 2C), and melanoma (MM) (FIG. 2D). Approximately half of the cell lines from each tumor type were relatively non-responsive to SSI-4 begging the question of whether there are common mechanisms for sensitivity and/or insensitivity to SSI-4.

FIGS. 3A-3C show soft agar growth of 1000 single cells from SSI-4 sensitive and SSI-4 insensitive cell lines plated on soft agar. Single cell replication and colony formation is an indication of an aggressive cancer cell with stem-like cell properties. SSI-4 sensitive cells lines form colonies on soft agar while insensitive cell lines do not (FIG. 3A). Sensitive cell lines were incubated with or without 1 micromolar SSI-4 and shown to be growth inhibited by SSI-4 (FIG. 3B). Quantitation of growth inhibition is shown in FIG. 3C.

FIGS. 4A-4D show SSI-4 specificity for targeting SCD1 by examining SSI-4 effects on cell proliferation, fatty acid synthesis, endoplasmic reticulum (ER) stress and apoptosis in SSI-4 responsive and SSI-4 non-responsive cell lines. FIG. 4A. Cell lines were incubated with DMSO control (1:1000), 500 μg/ml bovine serum albumin-oleic acid (BSA-OA) and/or 1 μM SSI-4 for four days and evaluated for cell viability. Nonresponsive cell lines are closed triangle and responsive lines are open triangles. OA is the end product of MUFA demonstrating complete rescue of SSI-4 inhibition of cell proliferation in responsive cell lines. FIG. 4B. Unsaturated fatty acids (UFAs) were measured in SSI-4 sensitive cells demonstrating elevated UFAs which were suppressed with 1 μM SSI-4 treatment that is equal to non-responsive cells which do not respond to 1 μm SSI-4. FIG. 4C. mRNA expression for ER stress induced proteins demonstrate that sensitive cells were elevated when exposed to 1 μM SSI-4. FIG. 4D Western blots shows ER stress (BIP), induction of apoptosis [cleaved PARP and cleaved caspase 3 (CC3)] and specificity for SCD1 blockade when cells are treated either with SSI-4 or OA.

FIGS. 5A-5B show energy phenotype and lactate synthesis in SSI-4 responsive and non-responsive cell lines. FIG. 5A. Using a Seahorse energy assay, most of the sensitive cell lines (open circles) fall into the glycolytic lower right quadrant. On the other hand, insensitive cells lines (closed circles) remained in the quiescent lower left quadrant. FIG. 5B. Secreted lactate was measured to determine glycolytic activity. Lactate was elevated in all four SSI-4 sensitive tumor type cell lines (open symbol) compared to non-responsive cells (closed symbols).

FIG. 6 shows that SCD1 protein is expressed in most cancer cells (HCC and ccRCC) while c-Myc protein expression appears to be expressed in SSI-4 sensitive cells. B-actin is protein loading control. p-Src, like cMyc, has been implicated in regulating glycolysis. pSrc and total Src protein were examined in cell lines. p-Src did not correlate with SSI-4 sensitive cell lines (HCC and RCC).

FIGS. 7A-7D show silencing of c-Myc in SSI-4 responsive cells down-regulated glycolytic genes and inhibited cell proliferation. FIG. 7A. c-Myc shRNA or nontarget shRNA were infected into cells. RT-PCR demonstrates 50-75% inhibition of c-Myc mRNA expression. FIG. 7B. Glycolytic genes SLC2A1 and LDHA are down-regulated with attenuation of c-Myc. FIG. 7C. c-Myc shRNA attenuates cell proliferation by 60-80% indicating that c-Myc is the predominant regulator of cell proliferation in c-Myc expressing cells. FIG. 7D. As seen in the main effects Plot, cells silenced for c-Myc become insensitive to SSI-4 treatment.

FIGS. 8A-8C show that overexpression of c-Myc in nonresponsive SSI-4 cells convert cells to SSI-4 sensitive (growth inhibited) cells. FIG. 8A. c-Myc mRNA expression is ˜2-fold elevated in cells infected with a c-Myc expression plasmid. FIG. 8B. Cell viability with empty vector and c-Myc expressing cells show no to slight difference in proliferation. FIG. 8C. As seen in the main effects Plot, cells overexpressing c-Myc become sensitive to SSI-4 treatment.

FIGS. 9A-9C show protein expression in appropriate ratios of SCD1, cMyc, LDHA and p-Src predict antitumor response of a SCD1 inhibitor in cancer cells. FIG. 9A. Table of IHC of formalin fixed paraffin embedded tumor tissues to assess the intensity level for SCD1, LDHA and phosphorylated-Src (p-Src) as well as percent of cells positive for c-Myc. SCD1 expression at any level is necessary but not predictive alone. FIG. 9B. Proliferation index of c-Myc with percent positivity of an 40% or greater is required for response to SCD1 inhibitor. FIG. 9C. Nuclear c-Myc protein expression in about 40% of cells or higher predicts response along with LDHA levels at an intensity of 3 or greater on a scale of 0-4+ intensity. High p-Src negatively predicts response. Levels of 1 or less when combined with high LDHA and c-Myc coupled with SCD1 expression predict antitumor activity of a SCD1 inhibitor. High p-Src negatively predicts response. Levels of p-Src (IHC score 0-1) when combined with high LDHA (IHC score 3+) and c-Myc (>40%) coupled with SCD1 (IHC score 1-3+) expression predict antitumor activity of a SCD1 inhibitor. p-Src (IHC score 2-3+), LDH (IHC score 0-2), c-Myc (<40% nuclear staining) or SCD1 (IHC=0) predict no response to SCD1 inhibitor. While all SSI-4 responsive cells must express SCD1, SCD1 expression alone is not sufficient to predict response to a SCD1 inhibitor such as SSI-4. Cells resistant to SSI-4 may have low or no cMyc, no or low LDHA or elevated p-Src. Cells with elevated cMyc and LDHA and low p-Src are responsive to SCD1 inhibition.

FIGS. 10A-10D show that PDX tumor models for RCC (FIG. 10A), HCC (FIG. 10B), melanoma (FIG. 10C), and CCA (FIG. 10D) respond to SSI-4 (180 parts per million in chow=10 mg/kg daily). Predictions were made prior to drug treatment from previously grown tumors that were formalin fixed, preserved in paraffin and slides prepared and stained using standard immunohistochemical (IHC) techniques for SCD1, c-Myc, LDHA and p-Src. PDTX tumors were grown in the right flank of NSG mice with treatment beginning when tumors reach ˜75 mm³ with the start of SSI-4 oral treatment. * indicates p<0.05 statistical significance.

FIG. 11 is a schematic showing exemplary markers for predicting SSI-4 responsiveness in SCD1 expressing cells. The presence of SCD1 protein is necessary (IHC score 0-4). C-Myc expression in the nucleus of cells should be ˜40% or higher for cells to respond. LDHA IHC score should be 3+ to predict a response to SCD1 inhibitor. p-Src levels should have a IHC score of 0-1 for cells to respond to SCD1 inhibitor. If any are out of range, the prediction is that cells will not respond to SCD1 inhibitor.

### DETAILED DESCRIPTION

This document provides methods and materials involved in assessing and/or treating mammals (e.g., humans) having cancer (e.g., a SCD1-associated cancer). In some cases, this document provides methods and materials for determining whether or not a mammal having cancer is likely to be responsive to one or more SCD1 polypeptide inhibitors (e.g., SSI-4), and, optionally, administering one or more cancer treatments that is/are selected based, at least in part, on whether or not the mammal is likely to be responsive to one or more SCD1 polypeptide inhibitors to the mammal. For example, a sample (e.g., a sample containing one or more cancer cells) obtained from a mammal (e.g., a human) having cancer can be assessed to determine if the mammal is likely to be responsive to one or more SCD1 polypeptide inhibitors (e.g., SSI-4) based, at least in part, on the presence, absence, or level of p-Src polypeptide expression in the sample. As described herein, the presence of a reduced level of p-Src polypeptide expression in a SCD1-associated cancer can indicate that the SCD1-associated cancer is likely to be responsive to treatment with one or more SCD1 polypeptide inhibitors (e.g., SSI-4). Also as described herein, the presence of an elevated level of polypeptide expression of one or more polypeptides that promote glycolysis, such as cMyc and LDHA, in a SCD1-associated cancer can indicate that the SCD1-associated cancer is likely to be responsive to treatment with one or more SCD1 polypeptide inhibitors (e.g., SSI-4). In some cases, this document provides methods and materials for treating a mammal having cancer (e.g., a SCD1-associated cancer) and/or identified as having a SCD1-associated cancer. For example, a mammal having a SCD1-associated cancer (e.g., a SCD1-associated cancer that is likely to be responsive to one or more SCD1 polypeptide inhibitors) can be administered one or more SCD1 polypeptide inhibitors to treat the mammal. For example, a mammal having a SCD1-associated cancer (e.g., a SCD1-associated cancer that is not likely to be responsive to one or more SCD1 polypeptide inhibitors) can be administered one or more alternative cancer treatments (e.g., cancer treatments other than one or more SCD1 polypeptide inhibitors) to treat the mammal.

Any appropriate mammal having a cancer can be assessed and/or treated as described herein. Examples of mammals having a cancer that can be assessed and/or treated as described herein include, without limitation, humans, non-human primates (e.g., monkeys), dogs, cats, horses, cows, pigs, sheep, mice, rats, gerbils, and guinea pigs. In some cases, a human having a cancer can be assessed and/or treated as described herein. For example, humans identified as having a SCD1-associated cancer can be assessed and/or treated as described herein.

When assessing and/or treating a mammal (e.g., a human) having a cancer as described herein, the cancer can be any type of cancer. For example, the cancer can be a SCD1-associated cancer. In some cases, a cancer can include one or more solid tumors. In some cases, a cancer can be a blood cancer. In some cases, a cancer can be a primary cancer. In some cases, a cancer can be a metastatic cancer. In some cases, a cancer can be a cancer that has escaped and/or has been non-responsive to chemotherapy (e.g., a chemoresistant cancer). Examples of cancers that can be treated as described herein include, without limitation, liver cancers (e.g., HCC and/or CCA), renal cell carcinomas (RCC), ovarian cancers, breast cancers, prostate cancers, colon cancers, pancreatic cancers, bladder cancers, lung cancers, thyroid cancers, melanomas, brain cancers, stomach cancers, cervical cancers, uterine cancers, chronic lymphocytic leukemias (CLLs), acute lymphocytic leukemias (ALLs), and lymphomas. In some cases, a cancer that can be assessed and/or treated as described herein can be as described elsewhere (see, e.g., von Roemeling et al., Oncotarget, 9(1):3-20 (2017)). In some cases, a cancer treated as described herein can be a HCC. In some cases, a cancer treated as described herein can be a CCA.

In some cases, the methods described herein can include identifying a mammal (e.g., a human) as having a cancer. Any appropriate method can be used to identify a mammal as having a cancer. For example, imaging techniques and/or biopsy techniques can be used to identify mammals (e.g., humans) having cancer.

In some cases, the methods described herein can include determining whether a cancer is a SCD1-associated cancer. Any appropriate method can be used to determine whether a mammal having cancer has a SCD1-associated cancer. For example, the presence, absence, or level of SCD1 polypeptide expression can be detected in a sample (e.g., a sample containing one or more cancer cells) obtained from a mammal to determine if the mammal has a SCD1-associated cancer. For example, when the presence of SCD1 polypeptide expression is detected, the mammal can be identified as having a SCD1-associated cancer.

A mammal having cancer (e.g., a SCD1-associated cancer) can be assessed to determine whether or not the cancer is likely to respond to one or more SCD1 polypeptide inhibitors (e.g., SSI-4). In some cases, a sample (e.g., a sample containing one or more cancer cells) obtained from a mammal having cancer can be assessed for the presence, absence, or level of p-Src polypeptide expression. As described herein, the level of p-Src polypeptide expression in a sample obtained from a mammal having a cancer can be used to determine whether or not the mammal is likely to respond to one or more SCD1 polypeptide inhibitors. For example, the presence of a decreased level of p-Src polypeptide expression in a sample obtained from a mammal having cancer can indicate that the mammal is likely to be responsive to one or more SCD1 polypeptide inhibitors. The term “decreased level” as used herein with respect to p-Src polypeptide expression refers to any level that is lower than a reference level of p-Src polypeptide expression. The term “reference level” as used herein with respect to p-Src polypeptide expression refers to the level of p-Src polypeptide expression typically observed in a sample (e.g., a control sample) from one or more healthy mammals (e.g., mammals that do not have a cancer). Control samples can include, without limitation, samples from normal (e.g., healthy) mammals, primary cell lines derived from normal (e.g., healthy mammals), and non-tumorigenic cells lines. In some cases, a decreased level of p-Src polypeptide expression can be a level that is at least 2 (e.g., at least 5, at least 10, at least 15, at least 20, at least 25, at least 35, or at least 50) fold lower relative to a reference level of p-Src polypeptide expression.

In some cases, a sample (e.g., a sample containing one or more cancer cells) obtained from a mammal having cancer can be assessed for the presence, absence, or level of polypeptide expression of one or more (e.g., one, two, three, four, five, or more) polypeptides that promote glycolysis. As described herein, the level of polypeptide expression of one or more polypeptides that promote glycolysis in a sample obtained from a mammal having a cancer can be used to determine whether or not the mammal is likely to respond to one or more SCD1 polypeptide inhibitors. For example, the presence of an elevated level of polypeptide expression of one or more polypeptides that promote glycolysis in a sample obtained from a mammal having cancer can indicate that the mammal is likely to be responsive to one or more SCD1 polypeptide inhibitors. Examples of polypeptides that promote glycolysis include, without limitation, cMyc, LDHA, glucose transporter 1 (GLUT1), hexokinase (HK2), phosphoglucose isomerase, phosphofructokinase, glyceraldehyde phosphate dehydrogenase, phosphoglycerate kinase, phosphoglyceromutase, enolase, and pyruvate kinase (PKM2). In some cases, a SCD1-associated cancer that is likely to be responsive to one or more SCD1 polypeptide inhibitors includes elevated levels of cMyc polypeptides. In some cases, a SCD1-associated cancer that is likely to be responsive to one or more SCD1 polypeptide inhibitors includes elevated levels of cMyc polypeptides and elevated levels of one or more polypeptides that promote glycolysis. The term “elevated level” as used herein with respect to polypeptide expression of a polypeptide that promotes glycolysis refers to any level that is higher than a reference level of polypeptide expression of the polypeptide that promotes glycolysis. The term “reference level” as used herein with respect to polypeptide of polypeptide that promotes glycolysis expression refers to the level of polypeptide expression or the polypeptide that promotes glycolysis typically observed in a sample (e.g., a control sample) from one or more healthy mammals (e.g., mammals that do not have a cancer). Control samples can include, without limitation, samples from normal (e.g., healthy) mammals, primary cell lines derived from normal (e.g., healthy mammals), and non-tumorigenic cells lines. In some cases, an elevated level of polypeptide expression of a polypeptide that promotes glycolysis can be a level that is at least 2 (e.g., at least 5, at least 10, at least 15, at least 20, at least 25, at least 35, or at least 50) fold greater relative to a reference level of polypeptide expression of the polypeptide that promotes glycolysis. In some cases, when control samples have an undetectable level of polypeptide expression of a polypeptide that promotes glycolysis, an elevated level can be any detectable level of polypeptide expression of the polypeptide that promotes glycolysis. It will be appreciated that levels from comparable samples are used when determining whether or not a particular level is an elevated level.

Any appropriate sample from a mammal (e.g., a human) having cancer can be assessed as described herein (e.g., for the presence, absence, or level of polypeptide expression of a SCD1 polypeptide, a p-Src polypeptide, a c-Myc polypeptide, and/or a LDHA polypeptide). In some cases, a sample can be a biological sample. In some cases, a sample can contain one or more cancer cells. In some cases, a sample can contain one or more biological molecules (e.g., nucleic acids such as DNA and RNA, polypeptides, carbohydrates, lipids, hormones, and/or metabolites). Examples of samples that can be assessed as described herein include, without limitation, tissue samples (e.g., tumor tissues such as those obtained by biopsy) such as liver tissue, fluid samples (e.g., whole blood, serum, plasma, urine, and saliva), and cellular samples (e.g., buccal samples). A sample can be a fresh sample or a fixed sample (e.g., a formaldehyde-fixed sample or a formalin-fixed sample). In some cases, a sample can be a processed sample (e.g., an embedded sample such as a paraffin or OCT embedded sample). In some cases, one or more biological molecules can be isolated from a sample. For example, nucleic acid (e.g., DNA and RNA such as messenger RNA (mRNA)) can be isolated from a sample and can be assessed as described herein. For example, one or more polypeptides can be isolated from a sample and can be assessed as described herein.

Any appropriate method can be used to detect the presence, absence, or level of polypeptide expression of one or more polypeptides (e.g., the presence, absence, or level of polypeptide expression of a SCD1 polypeptide, a p-Src polypeptide, a c-Myc polypeptide, and/or a LDHA polypeptide) within a sample (e.g., a sample containing one or more cancer cells) obtained from a mammal (e.g., a human). In some cases, the presence, absence, or level of polypeptide expression within a sample can be determined by detecting the presence, absence, or level of polypeptides in the sample. For example, immunoassays (e.g., immunohistochemistry (IHC) techniques and western blotting techniques), mass spectrometry techniques (e.g., proteomics-based mass spectrometry assays or targeted quantification-based mass spectrometry assays), enzyme-linked immunosorbent assays (ELISAs), and radio-immunoassays can be used to determine the presence, absence, or level of polypeptides in a sample. In some cases, the presence, absence, or level of polypeptide expression within a sample can be determined by detecting the presence, absence, or level of mRNA encoding a polypeptide in the sample. For example, polymerase chain reaction (PCR)-based techniques such as quantitative RT-PCR techniques, gene expression panel (e.g., next generation sequencing (NGS) such as RNA-seq), and in situ hybridization can be used to determine the presence, absence, or level of mRNA encoding a polypeptide in the sample.

In some cases, when an IHC technique is used to detect the presence, absence, or level of polypeptide expression of one or more polypeptides (e.g., the presence, absence, or level of polypeptide expression of a SCD1 polypeptide, a p-Src polypeptide, a c-Myc polypeptide, and/or a LDHA polypeptide) within a sample (e.g., a sample containing one or more cancer cells) obtained from a mammal (e.g., a human), the polypeptide being detected can be assigned an IHC intensity level (e.g., an IHC score). As used herein, an IHC intensity is assigned according to the percentage of the positively stained cells in the field of view. For example, on a scale of 0-4 intensity, when fewer than about 10% of cells in a field of view are positively stained cells an intensity level of 0 is assigned, when about 10% to about 25% of cells in a field of view are positively stained cells an intensity level of 1 is assigned, when about 25% to about 50% of cells in a field of view are positively stained cells an intensity level of 2 is assigned, when about 50% to about 75% of cells in a field of view are positively stained cells an intensity level of 3 is assigned, and when greater than about 75% of cells in a field of view are positively stained cells an intensity level of 4 is assigned. In some cases, an IHC intensity level of 0 or 1 can be used to detect a decreased level of p-Src polypeptide expression in a sample obtained from a mammal. In some cases, an IHC intensity level of 3 or 4 can be used to detect an elevated level of LDHA polypeptide expression in a sample obtained from a mammal.

In some cases, when an IHC technique is used to detect the presence, absence, or level of polypeptide expression of one or more polypeptides (e.g., the presence, absence, or level of polypeptide expression of a SCD1 polypeptide, a p-Src polypeptide, a c-Myc polypeptide, and/or a LDHA polypeptide) within a sample (e.g., a sample containing one or more cancer cells) obtained from a mammal (e.g., a human), nuclear staining can be used to detect presence, absence, or level of polypeptide expression. For example, when greater than about 40% (e.g., about 40%, about 45%, about 50%, about 55%, about 60%, about 65%, about 70%, about 75% or more) of cells in a field of view are positively stained with nuclear staining, the nuclear polypeptide can be an elevated level. In some cases, nuclear staining of C-Myc polypeptides in greater than about 40% of cells can be used to detect an elevated level of c-Myc polypeptide expression in a sample obtained from a mammal.

In some cases, a mammal (e.g., a human) having cancer (e.g., a SCD1-associated cancer) can be identified as being likely to respond to one or more SCD1 polypeptide inhibitors based, at least in part, on the presence of a decreased level of p-Src polypeptide expression, the presence of an elevated level of c-Myc polypeptide expression, and the presence of an elevated level of LDHA polypeptide expression within a sample (e.g., a sample containing one or more cancer cells) obtained from the mammal. For example, when a sample obtained from a mammal can be fixed in formalin, embedded in paraffin, and assessed for the presence of a decreased level of p-Src polypeptide expression, the presence of an elevated level of c-Myc polypeptide expression, and the presence of an elevated level of LDHA polypeptide expression in the sample using one or more IHC techniques. When a sample obtained from a mammal having a SCD1-associated cancer is assessed as described herein and is determined to have a p-Src polypeptide expression IHC intensity level of 0 or 1, is determined to have nuclear c-Myc polypeptide expression in greater than about 40% of cells, and is determined to have a LDHA polypeptide expression IHC intensity level of 3 or 4, the mammal can be identified as being likely to respond to one or more SCD1 polypeptide inhibitors.

In some cases, a sample (e.g., a sample containing one or more cancer cells) obtained from a mammal (e.g., a human) having cancer (e.g., a SCD1-associated cancer) can be assessed to determine if the mammal is likely to be responsive to one or more SCD1 polypeptide inhibitors (e.g., SSI-4) based, at least in part, on whether the sample can form one or more colonies in a soft agar assay. As described herein, the ability of a single cell from a SCD1-associated cancer plated on soft agar to form one or more colonies can indicate that the SCD1-associated cancer is likely to be responsive to treatment with one or more SCD1 polypeptide inhibitors (e.g., SSI-4). A sample that can be used in a soft agar assay can be a tissue sample obtained from a mammal (e.g., a human having cancer) such as surgical waste tissue or a biopsy tissue.

This document also provides methods and materials for treating mammals (e.g., humans) diagnosed with (or identified as having) a cancer (e.g., a SCD1-associated cancer). In some cases, a mammal having cancer (e.g., a SCD1-associated cancer) and assessed as described herein (e.g., to determine whether or not the cancer is likely to respond to one or more SCD1 polypeptide inhibitors based, at least in part, on the level of p-Src polypeptide expression), can be administered or instructed to self-administer any one or more (e.g., 1, 2, 3, 4, 5, 6, or more) cancer treatments, where the one or more cancer treatments are effective to treat the cancer within the mammal. For example, a mammal having cancer can be administered, or can be instructed to self-administer, any one or more cancer treatments that is/are selected based, at least in part, on whether or not the mammal is likely to be responsive to one or more SCD1 polypeptide inhibitors (e.g., based, at least in part, on the level of p-Src polypeptide expression). In some cases, the level of p-Src polypeptide expression within a sample (e.g., a sample containing one or more cancer cells) obtained from a mammal can be used to determine whether or not the mammal is likely to be responsive to one or more SCD1 polypeptide inhibitors. For example, the presence of a decreased level of p-Src polypeptide expression in a sample obtained from a mammal having a SCD1-associated cancer can indicate that the mammal is likely to be responsive to one or more SCD1 polypeptide inhibitors. For example, the absence of a decreased level of p-Src polypeptide expression (e.g., a normal level or reference level of p-Src polypeptide expression) in a sample obtained from a mammal having a SCD1-associated cancer can indicate that the mammal is not likely to be responsive to one or more SCD1 polypeptide inhibitors.

In some cases, one or more cancer treatments that is/are selected based, at least in part, on whether or not the mammal is likely to be responsive to one or more SCD1 polypeptide inhibitors (e.g., based, at least in part, on the level of p-Src polypeptide expression) can be administered to a mammal in need thereof (e.g., a mammal having a cancer such as a SCD1-associated cancer) to slow or prevent growth of a cancer. For example, one or more SCD1 polypeptide inhibitors described herein can be administered to a mammal having cancer as described herein to slow or prevent growth of a cancer within the mammal by, for example, 10, 20, 30, 40, 50, 60, 70, 80, 90, 95, or more percent. For example, administering one or more SCD1 polypeptide inhibitors to a mammal having a SCD1-associated cancer can be effective to prevent a tumor from increasing in size (e.g., volume). For example, administering one or more SCD1 polypeptide inhibitors to a mammal having a SCD1-associated cancer can be effective prevent a tumor from metastasizing.

In some cases, one or more cancer treatments that is/are selected based, at least in part, on whether or not the mammal is likely to be responsive to one or more SCD1 polypeptide inhibitors (e.g., based, at least in part, on the level of p-Src polypeptide expression) can be administered to a mammal in need thereof (e.g., a mammal having a cancer such as a SCD1-associated cancer) to reduce or eliminate the number of cancer cells within the mammal. For example, one or more SCD1 polypeptide inhibitors described herein can be administered to a mammal having cancer as described herein to reduce the number of cancer cells within the mammal by, for example, 10, 20, 30, 40, 50, 60, 70, 80, 90, 95, or more percent. For example, administering one or more SCD1 polypeptide inhibitors to a mammal having a SCD1-associated cancer can be effective to reduce the size (e.g., volume) of a tumor.

In some cases, one or more cancer treatments that is/are selected based, at least in part, on whether or not the mammal is likely to be responsive to one or more SCD1 polypeptide inhibitors (e.g., based, at least in part, on the level of p-Src polypeptide expression) can be administered to a mammal in need thereof (e.g., a mammal having a cancer such as a SCD1-associated cancer) to induce endoplasmic reticulum (ER) stress in cancer cells within the mammal.

In some cases, one or more cancer treatments that is/are selected based, at least in part, on whether or not the mammal is likely to be responsive to one or more SCD1 polypeptide inhibitors (e.g., based, at least in part, on the level of p-Src polypeptide expression) can be administered to a mammal in need thereof (e.g., a mammal having a cancer such as a SCD1-associated cancer) to induce apoptotic cell death of cancer cells within the mammal. For example, one or more SCD1 polypeptide inhibitors described herein can be administered to a mammal having cancer as described herein to increase the number of apoptotic cells within a tumor within the mammal by, for example, 10, 20, 30, 40, 50, 60, 70, 80, 90, 95, or more percent.

When treating a mammal (e.g., a human) having cancer (e.g., a SCD1-associated cancer) and identified as being likely to respond to one or more SCD1 polypeptide inhibitors as described herein (e.g., based, at least in part, on the presence of a decreased level of p-Src polypeptide expression), the mammal can be administered, or can be instructed to self-administer, any one or more (e.g., 1, 2, 3, 4, 5, 6, or more) SCD1 polypeptide inhibitors. A SCD1 polypeptide inhibitor can be any appropriate type of molecule (e.g., nucleic acid molecules designed to induce RNA interference (e.g., a siRNA molecule or a shRNA molecule), antisense molecules, miRNAs, and antibodies (e.g., antibodies (e.g., monoclonal antibodies)) that can reduce or eliminate SCD1 polypeptide expression or SCD1 polypeptide function. A SCD1 polypeptide inhibitor can be an inhibitor of SCD1 polypeptide expression or an inhibitor of SCD1 polypeptide activity. In some cases, a SCD1 polypeptide inhibitor can be readily designed based upon the nucleic acid and/or polypeptide sequences of SCD1. In some cases, a SCD1 polypeptide inhibitor can be as described elsewhere (see, e.g., WO 2016/022955). In some cases, a SCD1 polypeptide inhibitor can have Formula (I):

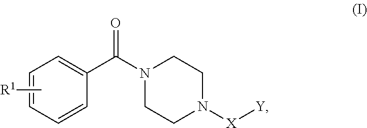

or a pharmaceutically acceptable salt thereof, where R¹ is an unsubstituted C₁₋₆alkyl or C₁₋₆haloalkyl; X is

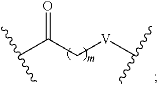

Y is selected from:

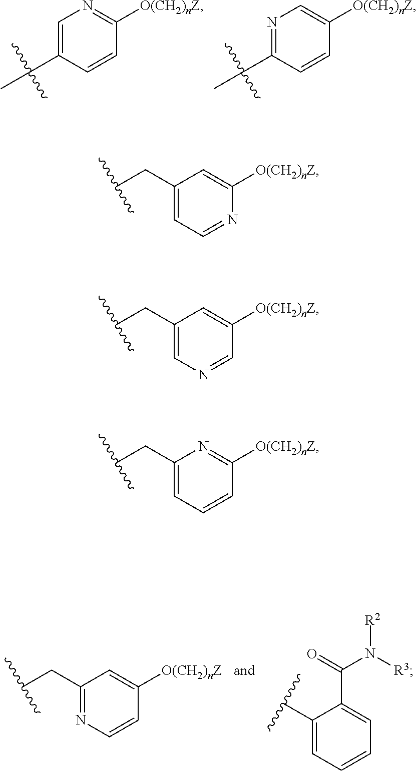

m is 0 or 1; n is 0, 1, or 2; V is NR⁴ or O; R², R³, and R⁴ are each independently H or an unsubstituted C₁₋₆alkyl; and Z is an unsubstituted aryl. In some cases, a SCD1 polypeptide inhibitor according to Formula (I) can have the structure of Formula (Ia):

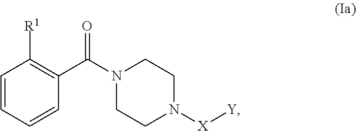

or a pharmaceutically acceptable salt thereof. Representative examples of SCD1 polypeptide inhibitors according to Formula (I) and/or Formula (Ia) include, without limitation:

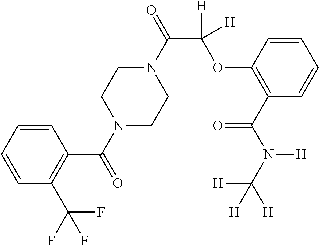 [SMILES: CNC(=O)c1ccccc1OCC(=O)N1CCN(C(=O)c2ccccc2C(F)(F)F)CC1]

SSI-1, N-Methyl-2-(2-oxo-2-{4-[2-(trifluoromethyl)benzoyl]piperazin-1-yl}ethoxy)benzamide;

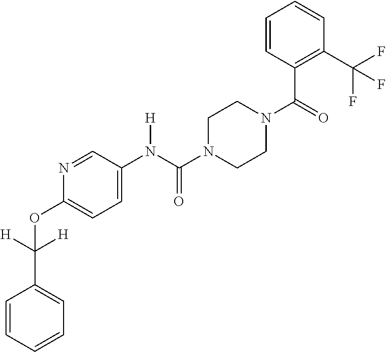 [SMILES: O=C(Nc1ccc(OCc2ccccc2)nc1)N1CCN(C(=O)c2ccccc2C(F)(F)F)CC1]

SSI-2,2-(benzyloxy)-5-{[hydroxy({4-[2-(trifluoromethyl)benzoyl]piperazin-1-yl})methyl]amino}-1,2-dihydropyridin-2-ylium-1-ide; and

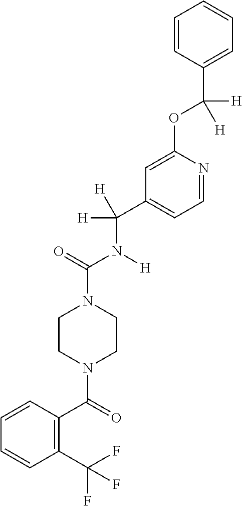 [SMILES: O=C(NCc1ccnc(OCc2ccccc2)c1)N1CCN(C(=O)c2ccccc2C(F)(F)F)CC1]

SSI-3, 2-(benzyloxy)-4-({[hydroxy({4-[2-(trifluoromethyl)benzoyl]piperazin-1-yl})methyl]azanidyl}methyl)-1,2-dihydropyridin-2-yliu m-1-ide, or a pharmaceutically acceptable salt thereof. In some cases, a SCD1 polypeptide inhibitor can have Formula (II):

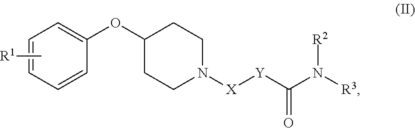

or pharmaceutically acceptable salt thereof, where R¹ is halo; X is —(C═O)NR⁴—; Y is

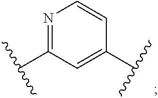 [SMILES: *c1ccnc(*)c1]

R², R³, and R⁴ are each independently H or an unsubstituted C₁₋₆alkyl.

In some cases, a SCD1 polypeptide inhibitor according to Formula (II) can have the structure of Formula (IIa):

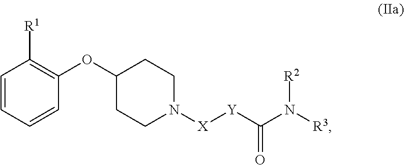

or pharmaceutically acceptable salt thereof. A representative example of a SCD1 polypeptide inhibitor according to Formula (II) and/or Formula (IIa) include:

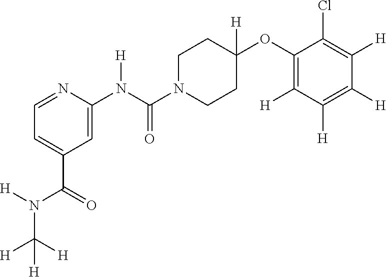 [SMILES: CNC(=O)c1ccnc(NC(=O)N2CCC(Oc3ccccc3Cl)CC2)c1]

SSI-4, 2-{[4-(2-Chlorophenoxy)piperidine-1-carbonyl]amino}-N-methylpyridine-4-carboxamide, or pharmaceutically acceptable salt thereof. In some cases, a SCD1 polypeptide inhibitor can be SSI-4.

In some cases, one or more SCD1 polypeptide inhibitors can be administered to a mammal (e.g., human) identified as having cancer (e.g., a SCD1-associated cancer) and identified as being likely to be responsive to one or more SCD1 polypeptide inhibitors as described herein as the sole active agent for treating the cancer. For example, SSI-4 can be administered to a mammal identified as having a SCD1-associated cancer and identified as being likely to be responsive to one or more SCD1 polypeptide inhibitors as described herein as the sole active agent for treating the cancer.

In some cases, one or more SCD1 polypeptide inhibitors can be administered to a mammal (e.g., human) identified as having cancer (e.g., a SCD1-associated cancer) and identified as being likely to be responsive to one or more SCD1 polypeptide inhibitors as described herein together with one or more additional treatments (e.g., therapeutic agents) used to treat cancer. In some cases, the one or more additional therapeutic agents can include agents approved by the Food and Drug Administration (FDA) for a particular type of cancer. For example, in cases where the mammal has a liver cancer, the one or more additional therapeutic agents can include agents approved by the FDA for liver cancer. Examples of cancer treatments that can be administered together with one or more SCD1 polypeptide inhibitors to a mammal having cancer and identified as being likely to be responsive to one or more SCD1 polypeptide inhibitors include, without limitation, radiation therapy; surgery; administering chemotherapeutic agents (including, but not limited to, mTOR inhibitors (e.g., temsirolimus and everolimus), proteosome inhibitors (e.g., bortezomib, carfilzomib, and ixazomib), immune checkpoint inhibitors (e.g., pembrolizumab, spartalizumab, atezolizumab), alkylating agents (e.g., cyclophosphamide, mechlorethamine, chlorambucil, melphalan, dacarbazine, nitrosoureas, temozolomide), anthracyclines (e.g., daunorubicin, doxorubicin, epirubicin, idarubicin, mitoxantrone, and valrubicin), cytoskeletal disruptors (e.g., paclitaxel, docetaxel, abraxane, and taxotere), histone deacetylase inhibitors (e.g., vorinostat and romidepsin), topoisomerase inhibitors (e.g., irinotecan, topotecan, etoposide, teniposide, and tafluposide), kinase inhibitors (e.g., sorafenib, regorafenib, bortezomib, erlotinib, gefitinib, imatinib, vemurafenib, and vismodegib), nucleotide analogs and precursor analogs (e.g., azacitidine, azathioprine, capecitabine, cytarabine, doxifluridine, fluorouracil, gemcitabine, hydroxyurea, mercaptopurine, methotrexate, and tioguanine), peptide antibiotics (e.g., bleomycin and actinomycin), platinum-based agents (e.g., carboplatin, cisplatin, and oxaliplatin), retinoids (e.g., tretinoin, alitretinoin, and bexarotene), and vinca alkaloids and derivatives (e.g., vinblastine, vincristine, vindesine, and vinorelbine)). For example, one or more SCD1 polypeptide inhibitors can be administered in combination with one or more mTOR inhibitors to a mammal (e.g., human) having a SCD1-associated RCC and identified as being likely to be responsive to one or more SCD1 polypeptide inhibitors to treat the mammal. For example, one or more SCD1 polypeptide inhibitors can be administered in combination with one or more proteosome inhibitors to a mammal (e.g., human) having a SCD1-associated thyroid cancer and identified as being likely to be responsive to one or more SCD1 polypeptide inhibitors to treat the mammal. For example, one or more SCD1 polypeptide inhibitors can be administered in combination with one or more immune checkpoint inhibitors to a mammal (e.g., human) having a SCD1-associated breast cancer and identified as being likely to be responsive to one or more SCD1 polypeptide inhibitors to treat the mammal. In cases where one or more SCD1 polypeptide inhibitors described herein are used in combination with one or more therapeutic agents to treat cancer, the one or more SCD1 polypeptide inhibitors can be administered at the same time or independently of the administration of one or more therapeutic agents. For example, the composition including one or more SCD1 polypeptide inhibitors can be administered before, concurrent with, or after the one or more therapeutic agents are administered.

In some cases, one or more SCD1 polypeptide inhibitors (e.g., SSI-4) can be administered to a mammal (e.g., a mammal identified as having a SCD1-associated cancer and/or identified as having a cancer that is likely to be responsive to one or more SCD1 polypeptide inhibitors) once or multiple times over a period of time ranging from days to weeks.

In some cases, one or more SCD1 polypeptide inhibitors described herein can be formulated into a pharmaceutically acceptable composition for administration to a mammal. For example, a therapeutically effective amount of one or more SCD1 polypeptide inhibitors can be formulated together with one or more pharmaceutically acceptable carriers (additives) and/or diluents. A pharmaceutical composition can be formulated for administration in solid or liquid form including, without limitation, sterile solutions, suspensions, sustained-release formulations, tablets, capsules, pills, powders, and granules. Pharmaceutically acceptable carriers, fillers, and vehicles that may be used in a pharmaceutical composition described herein include, without limitation, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

A pharmaceutical composition containing one or more SCD1 polypeptide inhibitors (e.g., SSI-4) can be designed for oral, parenteral (including subcutaneous, intramuscular, intravenous, and intradermal), or intratumoral administration. When being administered orally, a pharmaceutical composition can be in the form of a pill, tablet, or capsule. Compositions suitable for parenteral administration include aqueous and non-aqueous sterile injection solutions that can contain anti-oxidants, buffers, bacteriostats, and solutes that render the formulation isotonic with the blood of the intended recipient. The formulations can be presented in unit-dose or multi-dose containers, for example, sealed ampules and vials, and may be stored in a freeze dried (lyophilized) condition requiring only the addition of the sterile liquid carrier, for example, water for injections, immediately prior to use. Extemporaneous injection solutions and suspensions may be prepared from sterile powders, granules, and tablets.

In some cases, a pharmaceutically acceptable composition including one or more SCD1 polypeptide inhibitors (e.g., SSI-4) can be administered locally (e.g., intratumorally) or systemically. For example, a composition provided herein can be administered locally by injection into tumors or into biological spaces infiltrated by tumors (e.g. peritoneal cavity and/or pleural space). In some cases, a composition provided herein can be administered systemically, orally, or by injection to a mammal (e.g., a human).

Effective doses can vary depending on the risk and/or the severity of the cancer, the route of administration, the age and general health condition of the mammal, excipient usage, the possibility of co-usage with other therapeutic treatments such as use of other agents, and the judgment of the treating physician.

An effective amount of a composition containing one or more SCD1 polypeptide inhibitors (e.g., SSI-4) can be any amount that reduces the number of cancer cells present within the mammal without producing significant toxicity to the mammal. In some cases, an effective amount of one or more SCD1 polypeptide inhibitors can be from about 10 to about 500 mg per kg body weight (mg/kg) of the mammal being treated. For example, an effective amount of one or more SCD1 polypeptide inhibitors can be from about 10 to about 400, from about 10 to about 300, from about 10 to about 200, from about 10 to about 100, from about 10 to about 75, from about 10 to about 50, from about 10 to about 30, from about 25 to about 500, from about 50 to about 500, from about 100 to about 500, from about 200 to about 500, from about 300 to about 500, from about 15 to about 400, from about 20 to about 300, from about 25 to about 250, from about 30 to about 200, from about 35 to about 150, from about 40 to about 100, or from about 45 to about 75 mg/kg of the mammal being treated. In some cases, about 30 mg/kg of one or more SCD1 polypeptide inhibitors can be administered (e.g., orally administered) to a human identified as having a SCD1-associated cancer as described herein and/or identified as having a cancer that is likely to be responsive to one or more SCD1 polypeptide inhibitors as described herein. In some cases, about 50 mg/kg of one or more SCD1 polypeptide inhibitors can be administered (e.g., orally administered) to a human identified as having a SCD1-associated cancer as described herein and/or identified as having a cancer that is likely to be responsive to one or more SCD1 polypeptide inhibitors as described herein. In some cases, about 100 mg/kg of one or more SCD1 polypeptide inhibitors can be administered (e.g., orally administered) to a human identified as having a SCD1-associated cancer as described herein and/or identified as having a cancer that is likely to be responsive to one or more SCD1 polypeptide inhibitors as described herein.

If a particular mammal fails to respond to a particular amount, then the amount of one or more SCD1 polypeptide inhibitors (e.g., SSI-4) can be increased by, for example, two fold. After receiving this higher amount, the mammal can be monitored for both responsiveness to the treatment and toxicity symptoms, and adjustments made accordingly. The effective amount can remain constant or can be adjusted as a sliding scale or variable dose depending on the mammal's response to treatment. Various factors can influence the actual effective amount used for a particular application. For example, the frequency of administration, duration of treatment, use of multiple treatment agents, route of administration, and severity of the condition (e.g., cancer) may require an increase or decrease in the actual effective amount administered.

The frequency of administration of one or more SCD1 polypeptide inhibitors (e.g., SSI-4) can be any amount that reduces the number of cancer cells present within the mammal without producing significant toxicity to the mammal. For example, the frequency of administration of one or more SCD1 polypeptide inhibitors can be from about two to about three times a week to about two to about three times a month. In some cases, a mammal identified as having a SCD1-associated cancer and/or identified as having a cancer that is likely to be responsive to one or more SCD1 polypeptide inhibitors can receive a single administration of one or more SCD1 polypeptide inhibitors described herein. The frequency of administration of one or more SCD1 polypeptide inhibitors described herein can remain constant or can be variable during the duration of treatment. A course of treatment with a composition containing one or more SCD1 polypeptide inhibitors described herein can include rest periods. For example, a composition containing one or more SCD1 polypeptide inhibitors described herein can be administered every other month over a two-year period followed by a six-month rest period, and such a regimen can be repeated multiple times. As with the effective amount, various factors can influence the actual frequency of administration used for a particular application. For example, the effective amount, duration of treatment, use of multiple treatment agents, route of administration, and severity of the condition (e.g., cancer) may require an increase or decrease in administration frequency.

An effective duration for administering a composition containing one or more SCD1 polypeptide inhibitors (e.g., SSI-4) can be any duration that reduces the number of cancer cells present within the mammal without producing significant toxicity to the mammal. In some cases, the effective duration can vary from several months to several years. In general, the effective duration for reducing the number of cancer cells present within the mammal can range in duration from about one or two months to five or more years. Multiple factors can influence the actual effective duration used for a particular treatment. For example, an effective duration can vary with the frequency of administration, effective amount, use of multiple treatment agents, route of administration, and severity of the condition being treated.

When treating a mammal (e.g., a human) having cancer (e.g., a SCD1-associated cancer) and identified as not being likely to respond to one or more SCD1 polypeptide inhibitors as described herein (e.g., based, at least in part, on the absence of a decreased level of p-Src polypeptide expression), the mammal can be administered, or can be instructed to self-administer, any one or more (e.g., 1, 2, 3, 4, 5, 6, or more) alternative cancer treatments (e.g., cancer treatments other than one or more SCD1 polypeptide inhibitors). Examples of alternative cancer treatments that can administered to a mammal having a cancer and identified as not being likely to be responsive to one or more SCD1 polypeptide inhibitors include, without limitation, radiation therapy; surgery; administering chemotherapeutic agents (including, but not limited to, mTOR inhibitors (e.g., temsirolimus and everolimus), proteosome inhibitors (e.g., bortezomib, carfilzomib, and ixazomib), immune checkpoint inhibitors (e.g., pembrolizumab, spartalizumab, atezolizumab), alkylating agents (e.g., cyclophosphamide, mechlorethamine, chlorambucil, melphalan, dacarbazine, nitrosoureas, temozolomide), anthracyclines (e.g., daunorubicin, doxorubicin, epirubicin, idarubicin, mitoxantrone, and valrubicin), cytoskeletal disruptors (e.g., paclitaxel, docetaxel, abraxane, and taxotere), histone deacetylase inhibitors (e.g., vorinostat and romidepsin), topoisomerase inhibitors (e.g., irinotecan, topotecan, etoposide, teniposide, and tafluposide), kinase inhibitors (e.g., sorafenib, regorafenib, bortezomib, erlotinib, gefitinib, imatinib, vemurafenib, and vismodegib), nucleotide analogs and precursor analogs (e.g., azacitidine, azathioprine, capecitabine, cytarabine, doxifluridine, fluorouracil, gemcitabine, hydroxyurea, mercaptopurine, methotrexate, and tioguanine), peptide antibiotics (e.g., bleomycin and actinomycin), platinum-based agents (e.g., carboplatin, cisplatin, and oxaliplatin), retinoids (e.g., tretinoin, alitretinoin, and bexarotene), and vinca alkaloids and derivatives (e.g., vinblastine, vincristine, vindesine, and vinorelbine)). For example, one or more mTOR inhibitors to a mammal (e.g., human) having a SCD1-associated RCC and identified as not being likely to be responsive to one or more SCD1 polypeptide inhibitors to treat the mammal. For example, one or more proteosome inhibitors to a mammal (e.g., human) having a SCD1-associated thyroid cancer and identified as not being likely to be responsive to one or more SCD1 polypeptide inhibitors to treat the mammal. For example, one or more immune checkpoint inhibitors to a mammal (e.g., human) having a SCD1-associated breast cancer and identified as not being likely to be responsive to one or more SCD1 polypeptide inhibitors to treat the mammal.

The invention will be further described in the following examples, which do not limit the scope of the invention described in the claims.

### EXAMPLES

### Example 1: SCD1 Expression and SCD1 Inhibitor Response

Stearoyl CoA desaturase (SCD1), a key mediator of fatty acid (FA) biosynthesis and rate-limiting conversion of saturated fatty acids (SFAs) to mono-unsaturated fatty acids (MUFAs), is expressed in numerous cancers including renal cell carcinoma, hepatocellular carcinoma, cholangiocarcinoma and melanoma. Cancer cell lines derived from these four cancer types demonstrate that approximately half of the cell lines responded to SSI-4 with IC50 concentrations of 1-40 nM.

This Example demonstrates that SCD1 expression does not correlate with response to SCD1 inhibitor. While SCD1 expression is necessary, it is not sufficient to predict response to SCD1 inhibitor.

### Methods

Cells were seeded at 5,000 cells/well in clear-bottom 96-well plates in triplicate. Drug treatment was applied at 1:1000 in reduced serum conditions (3%). After 72 hours, cells were washed with PBS, and stored at −80° C. prior to analysis using CyQuant® Proliferation Analysis Kit (Invitrogen) per manufacturers' protocol for relative fluorescence units. Alternatively, cells were plated 2×10⁵/ well in 12-well plates (Genesee Scientific) in triplicate prior to drug treatment. After 120-hour treatment, cell number was established using a Coulter Particle Counter (Beckman). Oleic acid-albumin (Sigma Aldrich) was added to media at 5 μM, and was applied adjuvant to drug treatment. Drug stocks were prepared in DMSO (Sigma) at 1000×. IC50 dosing per cell line was calculated as described elsewhere (von Roemeling et al., Oncotarget, 9:3-20 (2018)).

Soft agar cultures were prepared by diluting 2× growth medium 1:1 in 1.5% SeaplaqueGTG agarose (Lonza), with 500 cells/plate in 60-mm culture dishes (Genesee Scientific). Colonies were stained with Giemsa (LabChem Inc.) and counted after 3 weeks.

### Results

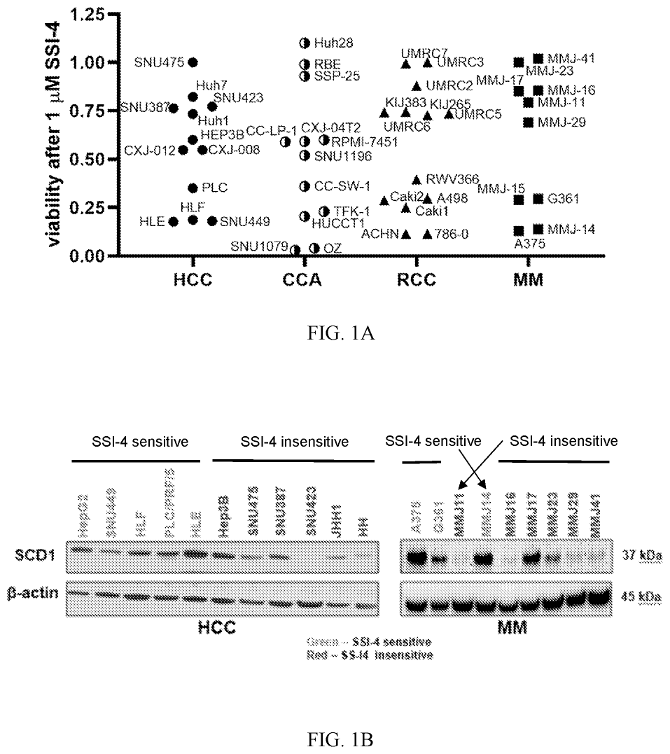

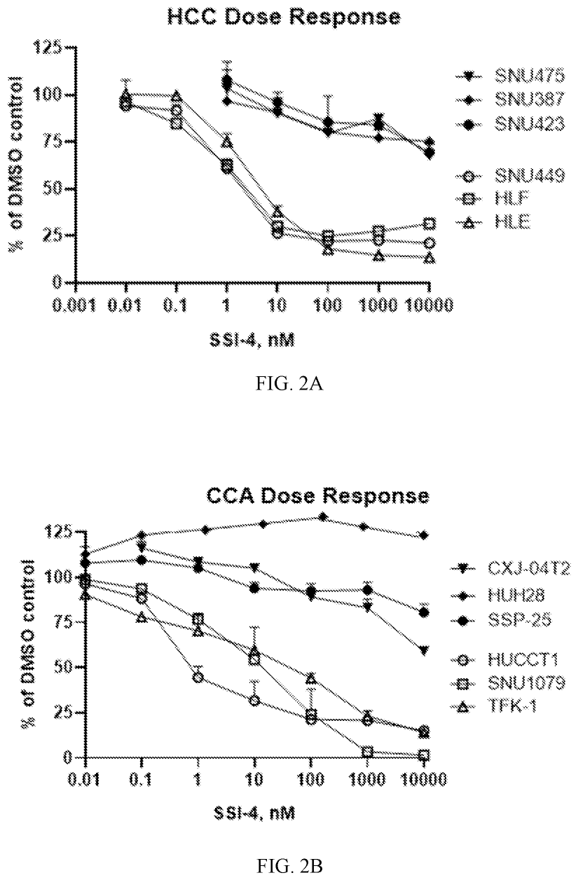

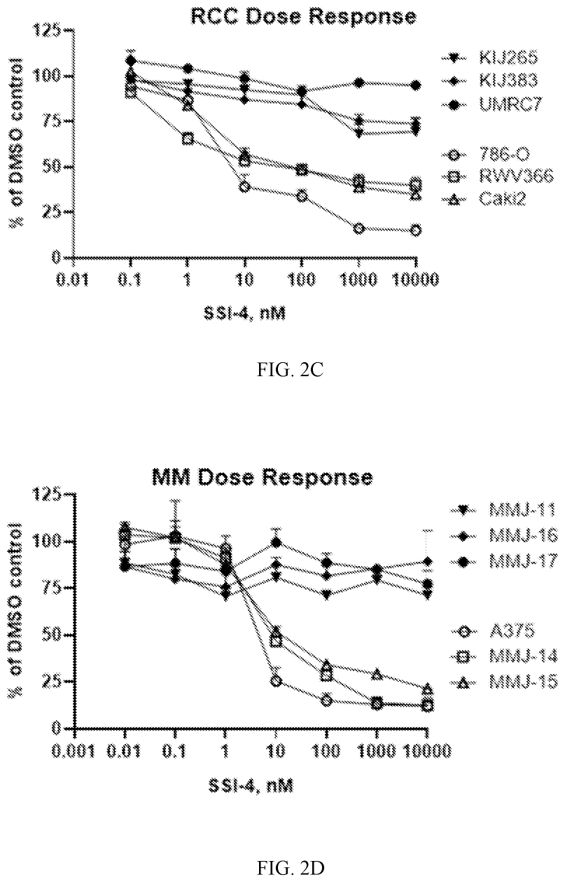

SCD1 expression was examined in cell lines. Cells were treated with 1 μM SSI-4 for 3 days and a viability assay was used to identify SSI-4 sensitive and insensitive cell lines. Certain cell lines were also treated with varied doses (0.01-10,000 nM) of SSI-4 for 3 days. Not all cells that express SCD1 are sensitive to a SCD1 inhibitor (FIG. 1 and FIG. 2). These results demonstrate that SCD1 expression alone cannot be used to predict tumor sensitivity to SCD1 inhibitors such as SSI-4.

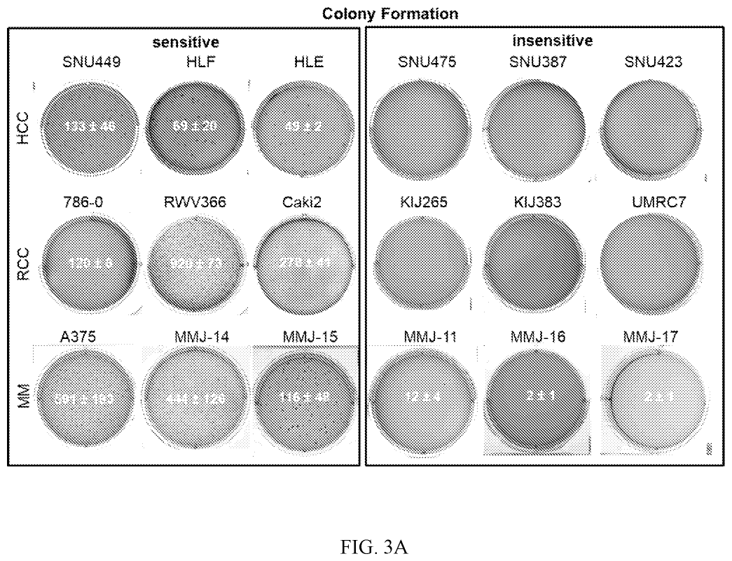

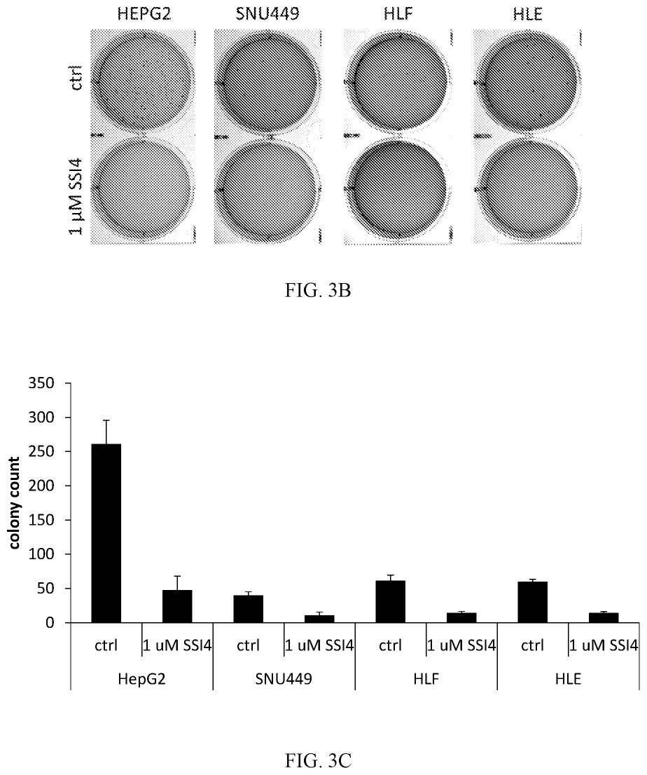

Soft agar growth of 1000 single cells plated on soft agar demonstrated that only SSI-4 sensitive cell lines grow on soft agar with insensitive growing not at all or very poorly (MM)(FIG. 3A). In FIG. 3A, cells representative of HCC (top row), RCC (middle row) and MM (bottom row) are shown. In FIG. 3B, sensitive HCC cells are treated with 1 micromolar SSI-4 showing inhibition of colony formation (i.e. growth). In FIG. 3C, colonies are quantitated comparing control to SSI-4 treated cells. SSI-4 blocked colony formation. Each treatment group was repeated in triplicate. Colony number is the mean+/−standard deviation (S.D.). Thus, soft agar assay can independently predict response to SCD1 inhibitor but requires fresh live tumor tissue and laboratories such as the Copland laboratory that can access tissues, process tissues and perform the assays. Furthermore, treatment of cells on soft agar with a SCD1 inhibitor will directly demonstrate response and sensitivity.

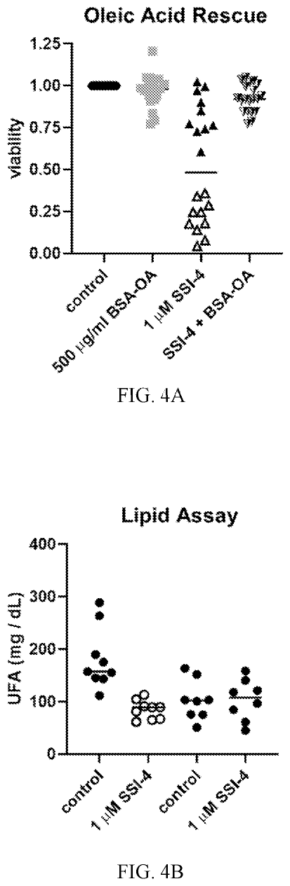

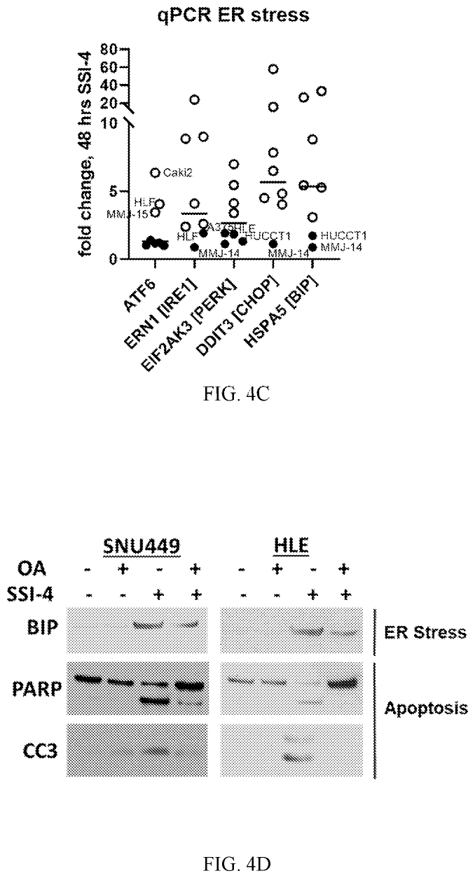

In FIG. 4, SSI-4 demonstrates specificity in SCD1 sensitive cell lines. HCC, RCC, CCA, and MM cell lines (2000 cells/well) were incubated with DMSO control (1:1000), 500 ug/ml bovine serum albumin-oleic acid (BSA-OA) and/or 1 μM SSI-4 for three days and determined for cell viability (FIG. 4A). Nonresponsive cell lines are closed triangle and responsive are open triangles. OA is the end product monounsaturated fatty acid (MUFA) demonstrating complete rescue of SSI-4 inhibition of cell proliferation in responsive cell lines when combined with SSI-4 (SSI-4+BSA-OA versus 1 μM SSI-4 open triangles). Unsaturated fatty acids (UFAs) were measured in media of SSI-4 sensitive cells demonstrating elevated UFAs compared to insensitive cell lines (FIG. 4B). 1 μM SSI-4 suppressed UFAs to levels equal to non-responsive cells which do not respond to 1 μm SSI-4. mRNA expression for endoplasmic reticulum (ER) stress induced proteins became elevated when exposed to 1 μM SSI-4 for 48 hours (FIG. 4C). mRNA expression was measured by qPCR and fold change to untreated cells was calculated. HCC cell lines respond to SSI-4 with ER stress induced as evidenced by upregulation of BIP protein as shown by Western analysis (FIG. 4D). Specificity for blockade of SCD1 is shown by rescue with the enzyme end product, oleic acid (OA). Apoptotic cell death is induced as shown by cleaved poly-ADP ribose polymerase (PARP) and cleaved caspase 3 (CC3). SSI-4 down-regulates cMyc protein with rescue by OA. Collectively, this data demonstrates 1) specificity of SSI-4 as a SCD1 inhibitor (oleic acid rescue FIG. 4A), 2) that unsaturated fatty content is elevated and suppressed by SSI-4 (FIG. 4B), and 3) SCD1 blockade leads to ER stress which then induces apoptotic cell death.

These results demonstrate that SCD1 expression alone cannot be used to predict tumor sensitivity to SCD1 inhibitors such as SSI-4.

### Example 2: cMyc Expression and Glycolysis Addiction and SCD1 Inhibitor Response

This Example demonstrates that silencing c-Myc downregulated key glycolysis genes rendered the cells resistant to SSI-4, while increased expression of c-Myc in SSI-4 resistant cell lines induced sensitization to SSI-4 growth inhibition. This Example also demonstrates that c-Myc polypeptides levels can be used to predict SSI-4 response in patient derived tumor xenograft (PDTX) animal models. Preclinical PDTX mouse models possess the highest predictability of patient response to therapy (80-85% predictive).

### Methods

Seahorse Energy Assay: Seahorse XF Analyzers measure oxygen consumption rate (OCR) and extracellular acidification rate (ECAR) of live cells in a multi-well plate, interrogating key cellular functions such as mitochondrial respiration and glycolysis. XF Analyzers perform compound addition and mixing, label-free analytical detection, and automatic measurement of OCR and ECAR in real time. Real-time measurements of OCR and ECAR are made by isolating an extremely small volume (about 2 μL) of medium above a monolayer of cells within a microplate. Cellular oxygen consumption (respiration) and proton excretion (glycolysis) cause rapid, easily measurable changes to the concentrations of dissolved oxygen and free protons in this “transient microchamber” which are measured every few seconds by solid state sensor probes residing 200 microns above the cell monolayer. The instrument measures the concentrations for 2-5 minutes then calculates the OCR and ECAR, respectively.

Lactate Assay: Harvest the amount of cells necessary for each assay (initial recommendation=2×10⁶ cells). Wash cells with cold PBS. Resuspend the cell pellet in 4× volumes of Lactate Assay Buffer (˜200 μL). Homogenize cells quickly by pipetting up and down a few times. Centrifuge 2-5 minutes at 4° C. at top speed in a cold microcentrifuge to remove any insoluble material. Collect supernatant and transfer to a clean tube. Keep on ice.

Cell samples may contain endogenous LDH that will degrade lactate. Remove enzyme from sample by using Deproteinizing Sample Preparation Kit—TCA. Set up Reaction wells and Reaction Mix. Mix and incubate at room temperature for 30 minutes. Measure output on a microplate reader at OD 450 nm.

c-Myc gene silencing (shRNA) and c-Myc over-expression: Lentivirus shRNA pLKO.1 constructs were used to make self-inactivating shRNA lentiviruses for human c-Myc and a nontarget random scrambled sequence control. Transfection reagents Lipofectamine 2000 and ViraPower were used to generate lentiviruses using HEK293FT viral progenitor cells. Cells were incubated with lentivirus plus 5 mg/mL polybrene for 24 hours before clonal selection with puromycin.

Immunohistochemistry (IHC): Samples include formalin-fixed, paraffin-embedded tumor tissue. Samples were mounted on slides, blocked with Diluent for 30 minutes, and then probed as specified in text for SCD1, c-Myc, LDHA, and p-Src. Stain scoring was done using algorithms generated with Imagescope software (Aperio) created by a histologist with scoring of 0-4). Nuclear scores were calculated by counting six different fields of 100 cells and determining the number of positive staining c-Myc expressing nuclei and determining the percent positive. 20× images were obtained using Scanscope XT and Imagescope software.

Western blot analysis: Cell protein extracts were prepared using RIPA lysis buffer containing 50 mmol/L Tris, 5 mmol/L EDTA, 150 mmol/L NaCl, 0.1% SDS, 0.5% deoxycholate, 1% NP40, protease inhibitor cocktail, and phosphatase inhibitor. Tissue protein extracts were prepared from frozen samples using 1% SDS in 50 mmol/L pH 8.0 Tris buffer containing protease inhibitor cocktail and phosphatase inhibitor with brief sonication on ice. Electrophoresis, transfer, blocking, and antibody preparations were performed. The following primary antibodies were included: SCD1, c-Myc, pSrc Y418, Src, b-actin, PARP, cleaved caspase 3, survivin, cyclin D1. Secondary species-specific horseradish peroxidase-labeled antibodies were applied, and Supersignal Chemiluminescent Kit was used to perform detection.

Animal models and drug testing: PDTX tumors (5×5×5-mm) were implanted into Nod Scid gamma mice with treatment (vehicle control or oral SSI-4) begun when tumors were approximately ˜75-100 mm³. Tumor volume, food consumption and body weight were monitored twice weekly. Tumor tissue was collected and processes for IHC.

DNA isolation and STR analysis of new cell lines and PDTX mouse models: Genomic DNA was extracted from cell lines and tumor tissues using Purelink Genomic DNA mini kit. Sixteen STR markers were PCR amplified using fluorescently labeled primers from ABI, and were analyzed using ABI 3130. Peak sizes were calculated versus a co-injected size standard using Gene Marker.

Statistical analysis: Data values are presented as either percentage or fold change +/−SD unless otherwise specified. Fold change values 1.5< are considered statistically significant. Treatment group comparisons were analyzed using 2-tailed paired Student t test with P<0.05 being considered statistically significant. Statistically significant results are indicated by asterisk (*).

### Results

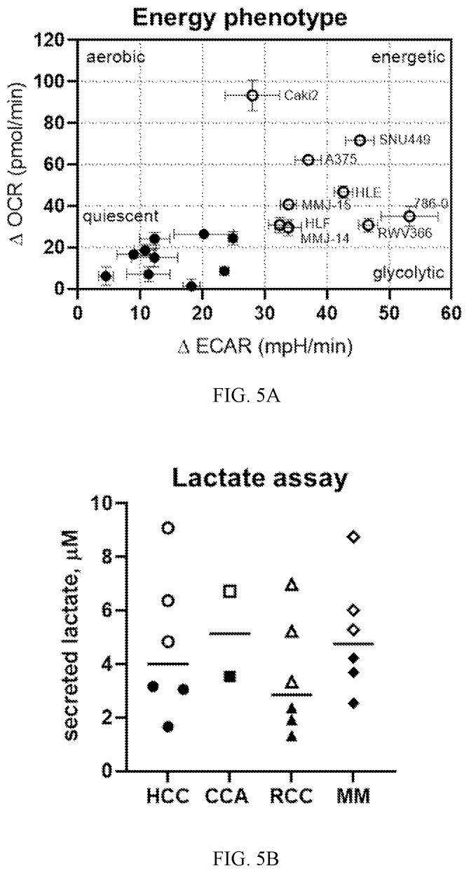

In FIG. 5, lactate synthesis and energy phenotype characterize SSI-4 responsive and non-responsive cell lines. Seahorse energy assay demonstrates that SSI-4 sensitive cells fall in the glycolytic lower right quadrant (open circles) while non-responsive cells are in the quiescent lower right quadrant (closed circles) (FIG. 5A). Secreted lactate was measured to determine glycolytic activity (FIG. 5B). Lactate was elevated in all four SSI-4 sensitive tumor type cell lines (open symbol) compared to non-responsive cells (closed symbols).

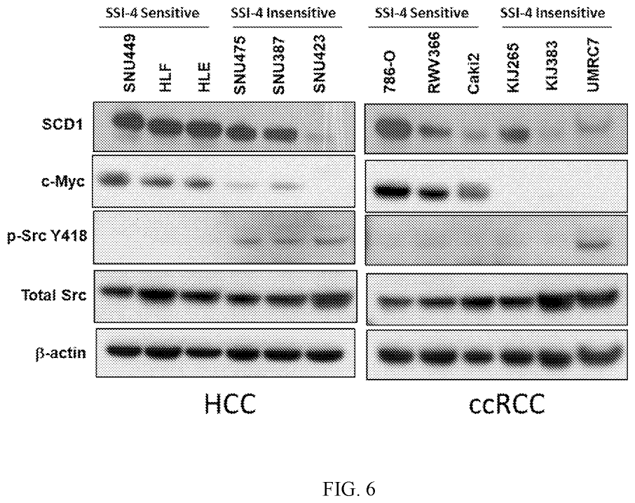

In FIG. 6, c-Myc protein expression correlates with SCD1 sensitive cell lines. SCD1 protein is expressed in most cancer cells (HCC and ccRCC) while c-Myc protein expression appears to be expressed in SSI-4 sensitive cells. In some cases, phosphorylated Src (p-Src) protein expression is inversely correlated with c-Myc expression. β-actin is protein loading control. c-Myc and c-Myc regulated genes (cyclin D1 and survivin) are down-regulated by 1 μM SSI-4 and restored by oleic acid BSA, thereby, demonstrating that SCD1 blockade down-regulates c-Myc and c-Myc dependent down-stream signaling i.e. survivin and cyclin D1. β-actin is used for protein loading control.

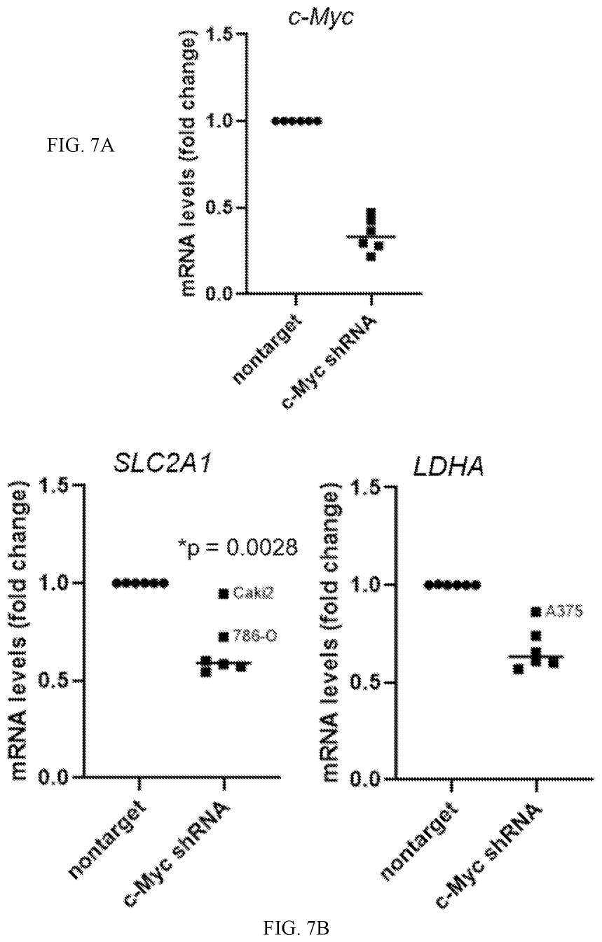

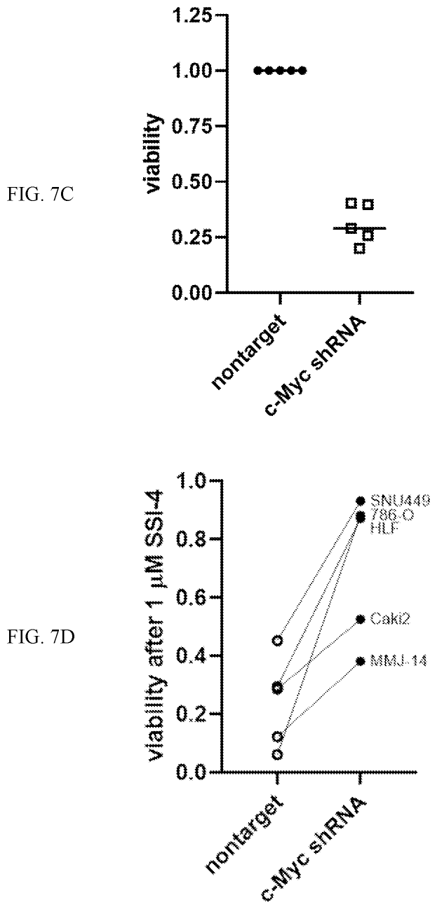

In FIG. 7, silencing of c-Myc in SSI-4 responsive cells leads to down-regulated glycolytic genes and inhibition of cell proliferation. c-Myc shRNA or nontarget shRNA were infected into cells. RT-PCR demonstrates 50-75% inhibition of c-Myc mRNA expression (FIG. 7A). Glycolytic genes SLC2A1 and LDHA known to be transcriptionally upregulated by c-Myc are down-regulated with attenuation of c-Myc (FIG. 7B). c-Myc shRNA attenuates cell proliferation by 60-80% indicating c-Myc may mediate SCD-1 regulated cell proliferation (FIG. 7C).

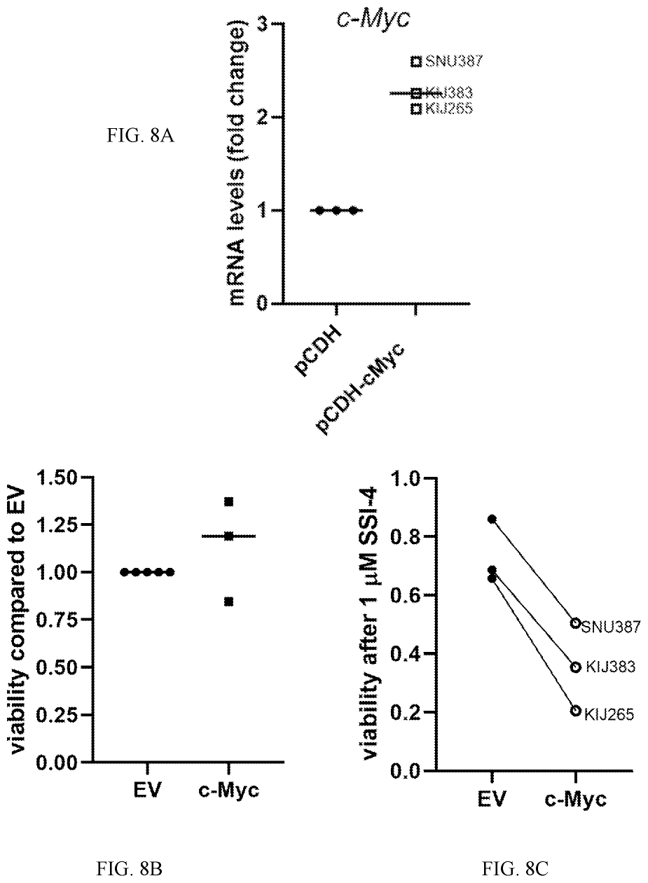

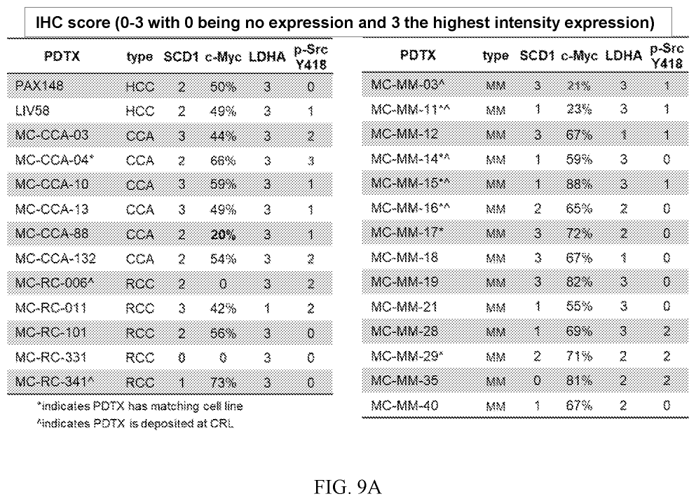

In FIG. 8, overexpression of c-Myc in nonresponsive SSI-4 cells convert cells to SSI-4 sensitive (growth inhibited) cells. c-Myc mRNA expression is ˜2-fold elevated in cells infected with a c-Myc expression plasmid (FIG. 8A). Cell viability with empty vector and c-Myc expressing cells shows no or slight difference in proliferation in c-Myc expressing cells (FIG. 8B). Treatment of c-Myc expressing cells with 1 uM SSI-4 leads to enhanced growth inhibition by 33-45%, thereby demonstrating that cells have become sensitized to SSI-4 (FIG. 8C).

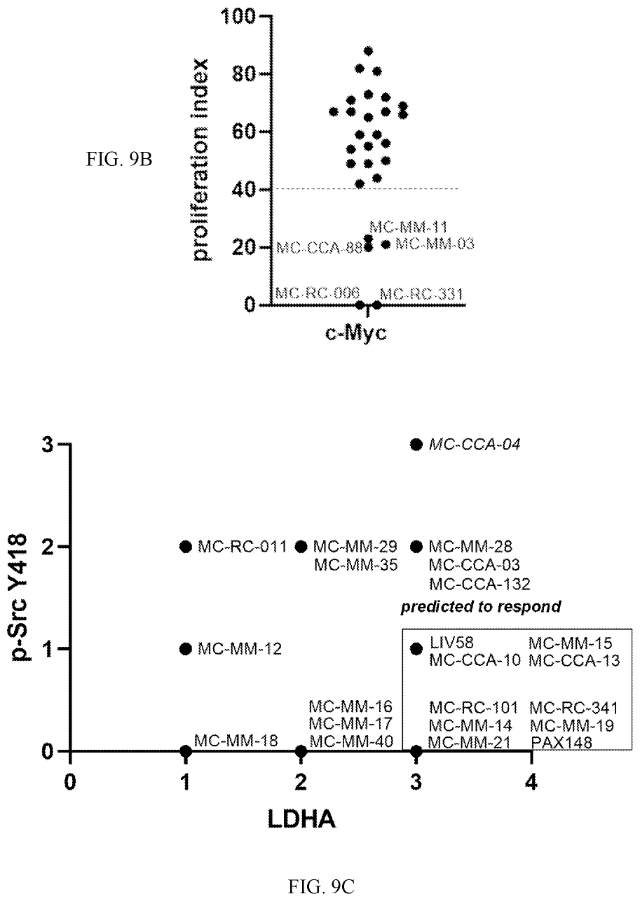

In FIG. 9, protein expression in appropriate ratios of SCD1, cMyc, LDHA and p-Src predict antitumor response of a SCD1 inhibitor in cancer cells. Immunohistochemical (IHC) of formalin fixed paraffin embedded tumor tissues can be used to assess the intensity level as well as percent of cells positive for SCD1, c-Myc, LDHA, and phophorylated-Src (p-Src) (FIG. 9A). SCD1 expression at any level is necessary but not predictive alone. Numbers bolded and in red predict no response to a SCD1 inhibitor. Nuclear c-Myc protein expression in about 40% of cells or higher predicts response along with LDHA levels at an intensity of 3 or greater on a scale of 0-4 intensity (FIG. 9B). High p-Src negatively predicts response. Levels of p-Src (IHC score 0-1) when combined with high LDHA (IHC score 3-4) and c-Myc (>40) coupled with SCD1 (IHC score 1-4) expression predict antitumor activity of a SCD1 inhibitor (FIG. 9C). p-Src (IHC score 2-4), LDH (IHC score 0-2), c-Myc (<40% nuclear staining), or SCD1 (IHC=0) predict no response to SCD1 inhibitor. PDX tumor models for HCC, ccRCC, CCA and melanoma respond to SSI-4. PDTX tumors were grown in the right flank of NSG mice with treatment beginning when tumors reach ˜75 mm³ with the start of SSI-4 oral treatment. Tumor volumes are statistically compared on Day 23 and paraffin block slides were stained for SCD1, c-Myc, LDHA, p-Src and scored. PDTX models tested include HCC, CCA, ccRCC, and melanoma. While all SSI-4 responsive cells must express SCD1, SCD1 expression alone is not sufficient to predict response to a SCD1 inhibitor such as SSI-4. Cells resistant to SSI-4 may have low or no cMyc, no or low LDHA or elevated p-Src. Cell with elevated cMyc and LDHA and low p-Src are responsive to SCD1 inhibition.

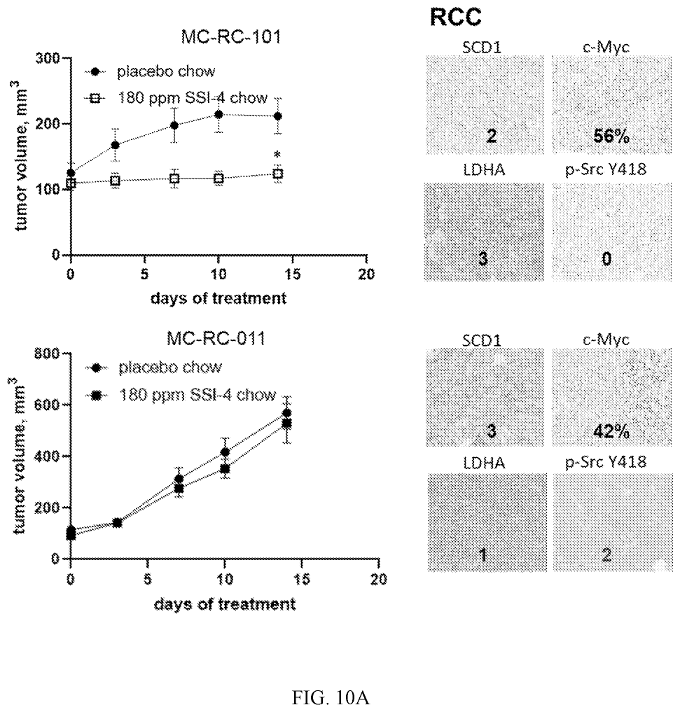

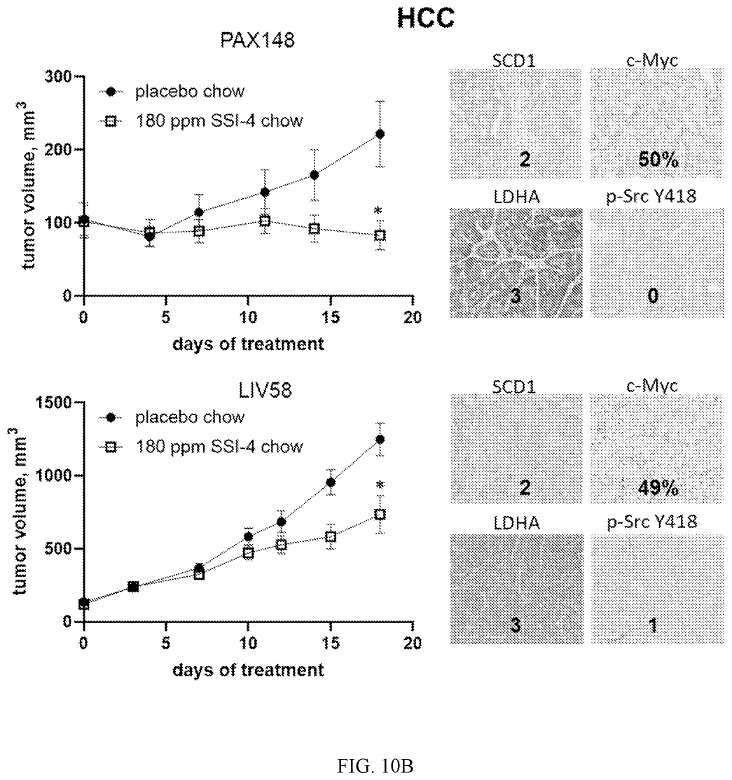

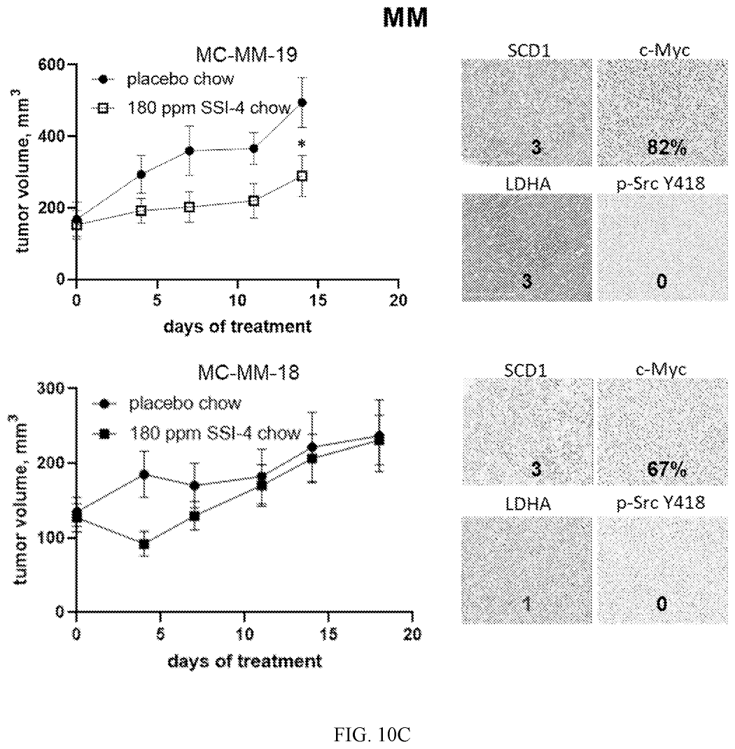

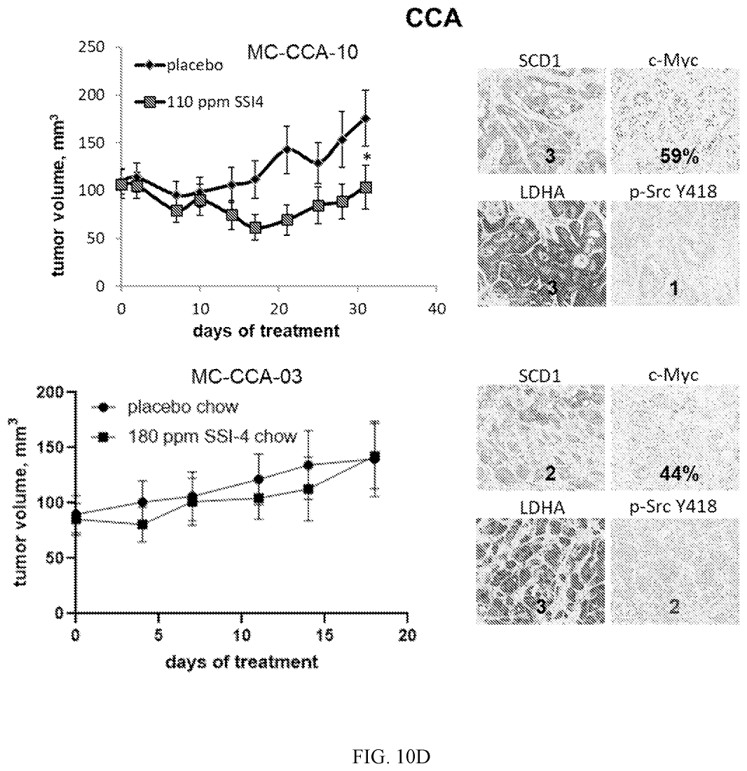

In FIG. 10, PDTX tumor models for HCC, ccRCC, CCA, and melanoma respond to SSI-4 or not correlate with predictions shown in FIG. 10. PDTX tumors were grown in the right flank of Nod Scid Gamma (NSG) mice with treatment beginning when tumors reach ˜75 mm³ with the start of SSI-4 oral treatment. Tumor tissues were formalin fixed and paraffin block slides were stained for SCD1, c-Myc, LDHA, p-Src by standard IHC protocols for protein expression. PDTX models tested include RCC, HCC, melanoma, and CCA. * indicates statistical difference of SSI-4 from same day treatment control (P<0.05). Number in red predicts no response to SSI-4 (SCD1 inhibitor). While all SSI-4 responsive cells must express SCD1, SCD1 expression alone does not predict response to a SCD1 inhibitor such as SSI-4. Cells resistant to SSI-4 may have low or no cMyc and no or low LDHA with elevated p-Src. Cell with elevated cMyc and LDHA and low p-Src are responsive to SSI-4 or SCD1 inhibition.

These results demonstrate that levels of p-Src polypeptides, levels of c-Myc polypeptides, and/or levels of LDHA polypeptides in SCD1-associated tumors can be used to predict tumor sensitivity to SSI-4.

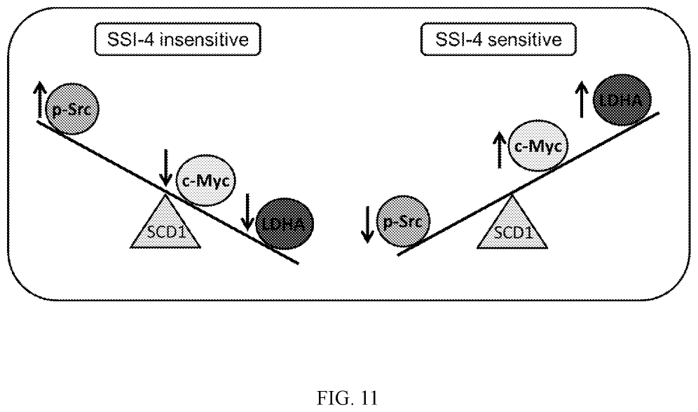

For example, FIG. 11 contains a schematic modeling exemplary markers for predicting SSI responsiveness. While all SSI-4 responsive cells must express SCD1, SCD1 expression alone does not predict response to a SCD1 inhibitor such as SSI-4. Cells resistant to SSI-4 may have low or no cMyc and no or low LDHA with elevated p-Src. Cell with elevated cMyc and LDHA and low p-Src are responsive to SSI-4 or SCD1 inhibition.

### Example 3: Exemplary Embodiments

Embodiment 1. A method for determining whether or not a mammal having a SCD1-associated cancer is likely to respond to treatment with a selective SCD1 inhibitor (SSI), wherein said method comprises:

(a) detecting a presence or absence of a decreased level of phosphorylated Src (p-Src) polypeptide expression in a sample from said mammal; and

(b) identifying said mammal as being likely to respond to said SSI if said presence of said decreased level is detected, or

(c) identifying said mammal as not being likely to respond to said SSI if said absence of said decreased level is detected.

Embodiment 2. The method of Embodiment 1, wherein said mammal is a human.
Embodiment 3. The method of any one of Embodiments 1-2, wherein said sample comprises cancer cells of said cancer.
Embodiment 4. The method of any one of Embodiments 1-3, wherein said cancer is a solid tumor, and wherein said cancer is selected from the group consisting of a liver cancer, a renal cell carcinoma, an ovarian cancer, a breast cancer, a prostate cancer, a colon cancer, a pancreatic cancer, a bladder cancer, a lung cancer, a thyroid cancer, a melanoma, a brain cancer, a stomach cancer, a cervical cancer, a uterine cancer, a chronic lymphocytic leukemia, a, acute lymphocytic leukemia, and a lymphoma.
Embodiment 5. The method of Embodiment 4, wherein said cancer is a liver cancer.
Embodiment 6. The method of Embodiment 5, wherein said liver cancer is a hepatocellular carcinoma.
Embodiment 7. The method of Embodiment 5, wherein said liver cancer is a cholangiocarcinoma.
Embodiment 8. The method of any one of Embodiments 1-7, further comprising detecting a presence or absence of an elevated level of c-Myc polypeptide expression in a sample from said mammal.
Embodiment 9. The method of any one of Embodiments 1-7, further comprising detecting a presence or absence of an elevated level of lactate dehydrogenase A (LDHA) polypeptide expression in a sample from said mammal.
Embodiment 10. The method of any one of Embodiments 8-9, wherein said method comprises detecting the presence of said decreased level of p-Src polypeptide expression, detecting the presence of said elevated level of c-Myc polypeptide expression, and detecting the presence of said elevated level of LDHA polypeptide expression, and wherein said method comprises identifying the mammal as being likely to respond to said SSI.
Embodiment 11. The method of Embodiment 10, wherein said detecting the presence of said decreased level of p-Src polypeptide expression, detecting the presence of said elevated level of c-Myc polypeptide expression, and detecting the presence of said elevated level of LDHA polypeptide expression comprises immunohistochemistry.
Embodiment 12. The method of Embodiment 11, wherein said decreased level of p-Src polypeptide expression comprises an IHC intensity level of 0 or 1, wherein said elevated level of c-Myc polypeptide expression comprises nuclear c-Myc polypeptide expression in greater than about 40% of cells in said sample, and said elevated level of LDHA polypeptide expression comprises an IHC intensity level of 3 or 4.
Embodiment 13. The method of any one of Embodiments 8-9, wherein said method comprises detecting the absence of said decreased level of p-Src polypeptide expression, detecting the absence of said elevated level of c-Myc polypeptide expression, and detecting the absence of said elevated level of LDHA polypeptide expression, and wherein said method comprises identifying the mammal as not being likely to respond to said SSI.
Embodiment 14. The method of any one of Embodiments 1-13, wherein said SCD1 polypeptide inhibitor is a compound having Formula (II) or Formula (IIa):

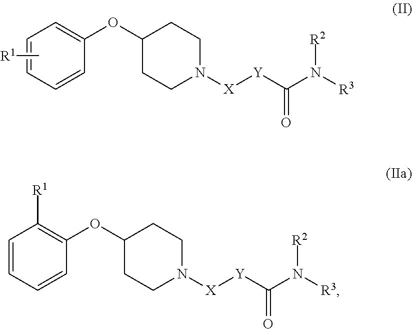

or a pharmaceutically acceptable salt thereof;
wherein:

R¹ is halo;

X is —(C═O)NR⁴—;

Y is

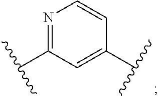 [SMILES: *c1ccnc(*)c1]

and

R², R³, and R⁴ are each independently H or an unsubstituted C₁₋₆alkyl.

Embodiment 15. The method of Embodiment 14, wherein said SCD1 polypeptide inhibitor is SSI-4, 2-{[4-(2-Chlorophenoxy)piperidine-1-carbonyl]amino}-N-methylpyridine-4-carboxamide:

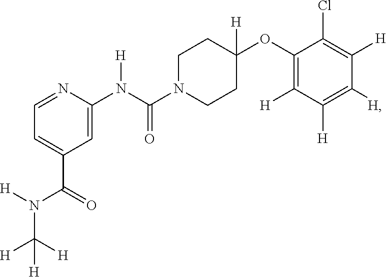

or a pharmaceutically acceptable salt thereof.
Embodiment 16. The method of any one of Embodiments 1-13, wherein said SCD1 polypeptide inhibitor
is a compound having Formula (I) or Formula (Ia):

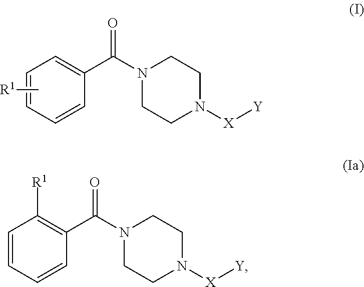

or a pharmaceutically acceptable salt thereof;
wherein:

R¹ is an unsubstituted C₁₋₆alkyl or C₁₋₆haloalkyl;

X is

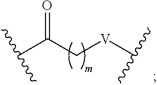

Y is selected from:

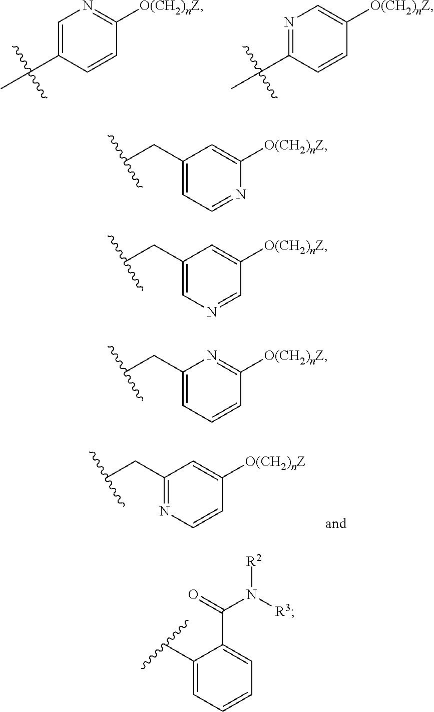

m is 0 or 1;

n is 0, 1, or 2;

V is NR⁴ or O;

R², R³, and R⁴ are each independently H or an unsubstituted C₁₋₆alkyl; and

Z is an unsubstituted aryl.

Embodiment 17. The method of Embodiment 16, wherein said SCD1 polypeptide inhibitor is SSI-2, 2-(benzyloxy)-5-{[hydroxy({4-[2-(trifluoromethyl)benzoyl]piperazin-1-yl})methyl]amino}-1,2-dihydropyridin-2-ylium-1-ide:

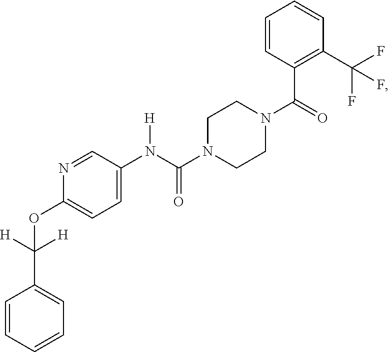 [SMILES: O=C(Nc1ccc(OCc2ccccc2)nc1)N1CCN(C(=O)c2ccccc2C(F)(F)F)CC1]

or a pharmaceutically acceptable salt thereof.
Embodiment 18. A method for treating a mammal having a SCD1-associated cancer, wherein said method comprises:

(a) detecting a decreased level of p-Src polypeptide expression in a sample obtained from said mammal; and

(b) administering a SCD1 polypeptide inhibitor to said mammal.

Embodiment 19. A method for treating a SCD1-associated cancer, wherein said method comprises administering a SCD1 polypeptide inhibitor to a mammal identified as having a decreased level of p-Src polypeptide expression in a sample obtained from said mammal.
Embodiment 20. The method of any one of Embodiments 18-19, wherein said mammal is a human.
Embodiment 21. The method of any one of Embodiments 18-20, wherein said sample comprises cancer cells of said cancer.
Embodiment 22. The method of any one of Embodiments 18-21, wherein said cancer is a solid tumor, and wherein said cancer is selected from the group consisting of a liver cancer, a renal cell carcinoma, an ovarian cancer, a breast cancer, a prostate cancer, a colon cancer, a pancreatic cancer, a bladder cancer, a lung cancer, a thyroid cancer, a melanoma, a brain cancer, a stomach cancer, a cervical cancer, a uterine cancer, a chronic lymphocytic leukemia, a, acute lymphocytic leukemia, and a lymphoma.
Embodiment 23. The method of Embodiment 22, wherein said cancer is a liver cancer.
Embodiment 24. The method of Embodiment 23, wherein said liver cancer is a hepatocellular carcinoma.
Embodiment 25. The method of Embodiment 23, wherein said liver cancer is a cholangiocarcinoma.
Embodiment 26. The method of any one of Embodiments 18-25, further comprising detecting an elevated level of c-Myc polypeptide expression in a sample from said mammal.
Embodiment 27. The method of any one of Embodiments 18-26, further comprising detecting an elevated level of LDHA polypeptide expression in a sample from said mammal.
Embodiment 28. The method of any one of Embodiments 26-27, wherein said method comprises detecting the presence of said decreased level of p-Src polypeptide expression, detecting the presence of said elevated level of c-Myc polypeptide expression, and detecting the presence of said elevated level of LDHA polypeptide expression.
Embodiment 29. The method of Embodiment 28, wherein said detecting the presence of said decreased level of p-Src polypeptide expression, detecting the presence of said elevated level of c-Myc polypeptide expression, and detecting the presence of said elevated level of LDHA polypeptide expression comprises immunohistochemistry.
Embodiment 30. The method of Embodiment 29, wherein said decreased level of p-Src polypeptide expression comprises an IHC intensity level of 0 or 1, wherein said elevated level of c-Myc polypeptide expression comprises nuclear c-Myc polypeptide expression in greater than about 40% of cells in said sample, and said elevated level of LDHA polypeptide expression comprises an IHC intensity level of 3 or 4.
Embodiment 31. The method of any one of Embodiments 18-30, wherein said SCD1 polypeptide inhibitor is a compound having Formula (II) or Formula (IIa):

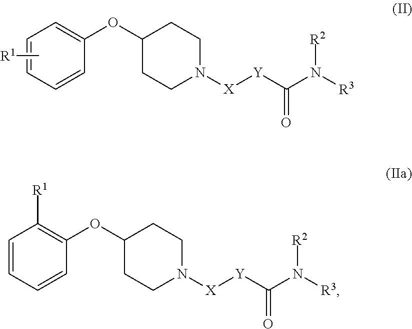

or a pharmaceutically acceptable salt thereof;
wherein:

R¹ is halo;

X is —(C═O)NR⁴—;

Y is

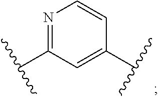 [SMILES: *c1ccnc(*)c1]

and

R², R³, and R⁴ are each independently H or an unsubstituted C₁₋₆alkyl.

Embodiment 32. The method of Embodiment 31, wherein said SCD1 polypeptide inhibitor is SSI-4, 2-{[4-(2-Chlorophenoxy)piperidine-1-carbonyl]amino}-N-methylpyridine-4-carboxamide:

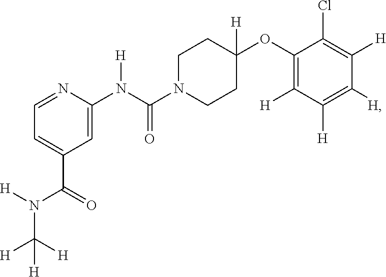

or a pharmaceutically acceptable salt thereof.
Embodiment 33. The method of any one of Embodiments 18-30, wherein said SCD1 polypeptide inhibitor
is a compound having Formula (I) or Formula (Ia):

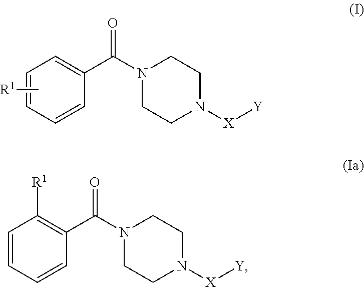

or a pharmaceutically acceptable salt thereof;
wherein:

R¹ is an unsubstituted C₁₋₆alkyl or C₁₋₆haloalkyl;

X is

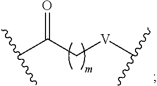

Y is selected from:

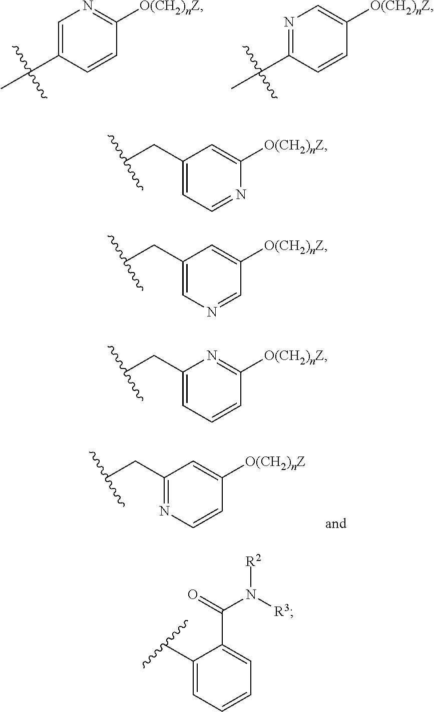

m is 0 or 1;

n is 0, 1, or 2;

V is NR⁴ or O;

R², R³, and R⁴ are each independently H or an unsubstituted C₁₋₆alkyl; and

Z is an unsubstituted aryl.

Embodiment 34. The method of Embodiment 33, wherein said SCD1 polypeptide inhibitor is SSI-2, 2-(benzyloxy)-5-{[hydroxy({4-[2-(trifluoromethyl)benzoyl]piperazin-1-yl})methyl]amino}-1,2-dihydropyridin-2-ylium-1-ide:

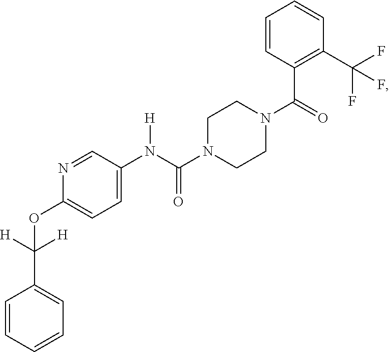 [SMILES: O=C(Nc1ccc(OCc2ccccc2)nc1)N1CCN(C(=O)c2ccccc2C(F)(F)F)CC1]

or a pharmaceutically acceptable salt thereof.
Embodiment 35. The method of any one of Embodiments 18-34, wherein said method further comprises administering a cancer treatment to said mammal.
Embodiment 36. The method of Embodiment 35, wherein said cancer treatment comprises a kinase inhibitor.
Embodiment 37. The method of Embodiment 36, wherein said kinase inhibitor is regorafenib.
Embodiment 38. The method of Embodiment 35, wherein said cancer treatment comprises a mTOR inhibitor.
Embodiment 39. The method Embodiment 35 wherein said cancer treatment comprises a proteosome inhibitor.
Embodiment 40. The method of Embodiment 35, wherein said cancer treatment comprises an immune checkpoint inhibitor.
Embodiment 41. A method for treating a mammal having a SCD1-associated cancer, wherein said method comprises:

(a) detecting an absence of a decreased level of p-Src polypeptide expression in a sample obtained from said mammal; and

(b) administering a cancer treatment to said mammal, wherein said cancer treatment is not a SCD1 polypeptide inhibitor.

Embodiment 42. A method for treating a SCD1-associated cancer, wherein said method comprises administering a SCD1 polypeptide inhibitor to a mammal identified as having an absence of a decreased level of p-Src polypeptide expression in a sample obtained from said mammal.
Embodiment 43. The method of any one of Embodiments 41-42, wherein said mammal is a human.
Embodiment 44. The method of any one of Embodiments 41-43, wherein said sample comprises cancer cells of said cancer.
Embodiment 45. The method of any one of Embodiments 41-44, wherein said cancer is a solid tumor, and wherein said cancer is selected from the group consisting of a liver cancer, a renal cell carcinoma, an ovarian cancer, a breast cancer, a prostate cancer, a colon cancer, a pancreatic cancer, a bladder cancer, a lung cancer, a thyroid cancer, a melanoma, a brain cancer, a stomach cancer, a cervical cancer, a uterine cancer, a chronic lymphocytic leukemia, a, acute lymphocytic leukemia, and a lymphoma.
Embodiment 46. The method of Embodiment 45, wherein said cancer is a liver cancer.
Embodiment 47. The method of Embodiment 46, wherein said liver cancer is a hepatocellular carcinoma.
Embodiment 48. The method of Embodiment 46, wherein said liver cancer is a cholangiocarcinoma.
Embodiment 49. The method of any one of Embodiments 41-48, further comprising detecting an absence of an elevated level of c-Myc polypeptide expression in a sample from said mammal.
Embodiment 50. The method of any one of Embodiments 41-48, further comprising detecting an absence of an elevated level of LDHA polypeptide expression in a sample from said mammal.
Embodiment 51. The method of any one of Embodiments 49-50, wherein said method comprises detecting the absence of said decreased level of p-Src polypeptide expression, detecting the absence of said elevated level of c-Myc polypeptide expression, and detecting the absence of said elevated level of LDHA polypeptide expression.
Embodiment 52. The method of any one of Embodiments 41-51, wherein said cancer treatment is surgery.
Embodiment 53. The method of any one of Embodiments 41-51, wherein said cancer treatment is radiation therapy.
Embodiment 54. A method for determining whether or not a mammal having a SCD1-associated cancer is likely to respond to treatment with a selective SCD1 inhibitor (SSI), wherein said method comprises:

(a) plating a cell from a sample from said mammal on soft agar; and

(b) identifying said mammal as being likely to respond to said SSI if said cell forms a colony in said soft agar, or

(c) identifying said mammal as not being likely to respond to said SSI if said cell does not form a colony in said soft agar.

Embodiment 55. The method of Embodiment 54, wherein said mammal is a human.
Embodiment 56. The method of any one of Embodiments 54-55, wherein said sample comprises cancer cells of said cancer.
Embodiment 57. The method of any one of Embodiments 54-56, wherein said cancer is a solid tumor, and wherein said cancer is selected from the group consisting of a liver cancer, a renal cell carcinoma, an ovarian cancer, a breast cancer, a prostate cancer, a colon cancer, a pancreatic cancer, a bladder cancer, a lung cancer, a thyroid cancer, a melanoma, a brain cancer, a stomach cancer, a cervical cancer, a uterine cancer, a chronic lymphocytic leukemia, a, acute lymphocytic leukemia, and a lymphoma.
Embodiment 58. The method of Embodiment 57, wherein said cancer is a liver cancer.
Embodiment 59. The method of Embodiment 58, wherein said liver cancer is a hepatocellular carcinoma.
Embodiment 60. The method of Embodiment 58, wherein said liver cancer is a cholangiocarcinoma.
Embodiment 61. A method for treating a mammal having a SCD1-associated cancer, wherein said method comprises:

(a) detecting a presence of soft agar colony formation from a cell from a sample obtained from said mammal; and

(b) administering a SCD1 polypeptide inhibitor to said mammal.

Embodiment 62. A method for treating a SCD1-associated cancer, wherein said method comprises administering a SCD1 polypeptide inhibitor to a mammal identified as having a presence of soft agar colony formation from a cell from a sample obtained from said mammal.
Embodiment 63. The method of any one of Embodiments 61-62, wherein said mammal is a human.
Embodiment 64. The method of any one of Embodiments 61-63, wherein said sample comprises cancer cells of said cancer.
Embodiment 65. The method of any one of Embodiments 61-64, wherein said cancer is a solid tumor, and wherein said cancer is selected from the group consisting of a liver cancer, a renal cell carcinoma, an ovarian cancer, a breast cancer, a prostate cancer, a colon cancer, a pancreatic cancer, a bladder cancer, a lung cancer, a thyroid cancer, a melanoma, a brain cancer, a stomach cancer, a cervical cancer, a uterine cancer, a chronic lymphocytic leukemia, a, acute lymphocytic leukemia, and a lymphoma.
Embodiment 66. The method of Embodiment 65, wherein said cancer is a liver cancer.
Embodiment 67. The method of Embodiment 66, wherein said liver cancer is a hepatocellular carcinoma.
Embodiment 68. The method of Embodiment 66, wherein said liver cancer is a cholangiocarcinoma.
Embodiment 69. The method of any one of Embodiments 54-68, wherein said SCD1 polypeptide inhibitor is a compound having Formula (II) or Formula (IIa):

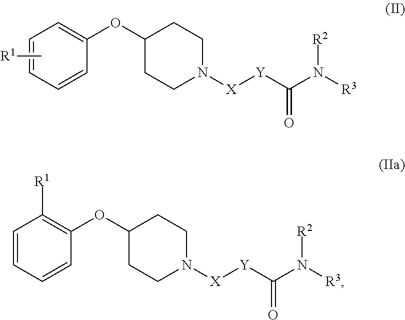

or a pharmaceutically acceptable salt thereof;
wherein:

R¹ is halo;

X is —(C═O)NR⁴—;

Y is

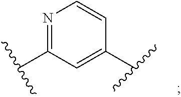 [SMILES: *c1ccnc(*)c1]

and

R², R³, and R⁴ are each independently H or an unsubstituted C₁₋₆alkyl.

Embodiment 70. The method of Embodiment 69, wherein said SCD1 polypeptide inhibitor is SSI-4, 2-{[4-(2-Chlorophenoxy)piperidine-1-carbonyl]amino}-N-methylpyridine-4-carboxamide:

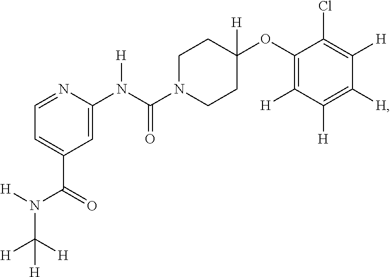

or a pharmaceutically acceptable salt thereof.
Embodiment 71. The method of any one of Embodiments 54-68, wherein said SCD1 polypeptide inhibitor
is a compound having Formula (I) or Formula (Ia):

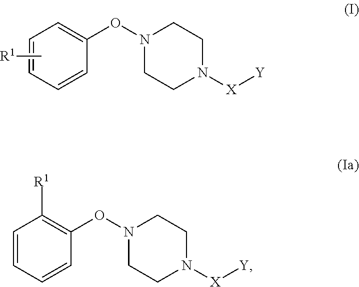

or a pharmaceutically acceptable salt thereof;
wherein:

R¹ is an unsubstituted C₁₋₆alkyl or C₁₋₆haloalkyl;

X is

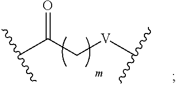

Y is selected from:

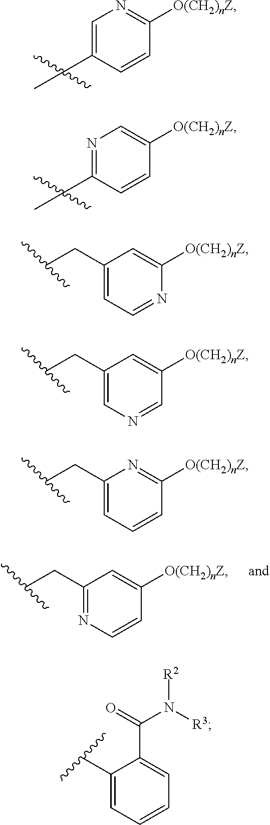

m is 0 or 1;

n is 0, 1, or 2;

V is NR⁴ or O;

R², R³, and R⁴ are each independently H or an unsubstituted C₁₋₆alkyl; and

Z is an unsubstituted aryl.

Embodiment 72. The method of Embodiment 71, wherein said SCD1 polypeptide inhibitor is SSI-2, 2-(benzyloxy)-5-{[hydroxy({4-[2-(trifluoromethyl)benzoyl]piperazin-1-yl})methyl]amino}-1,2-dihydropyridin-2-ylium-1-ide:

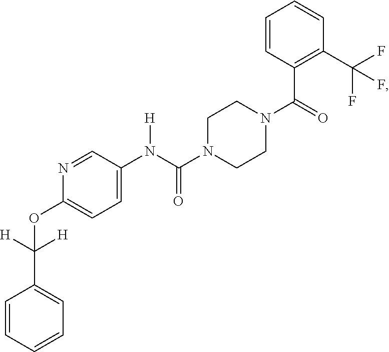 [SMILES: O=C(Nc1ccc(OCc2ccccc2)nc1)N1CCN(C(=O)c2ccccc2C(F)(F)F)CC1]

or a pharmaceutically acceptable salt thereof.
Embodiment 73. The method of any one of Embodiments 60-72, wherein said method further comprises administering a cancer treatment to said mammal.
Embodiment 74. The method of Embodiment 73, wherein said cancer treatment comprises a kinase inhibitor.
Embodiment 75. The method of Embodiment 74, wherein said kinase inhibitor is regorafenib.
Embodiment 76. The method of Embodiment 73, wherein said cancer treatment comprises a mTOR inhibitor.
Embodiment 77. The method Embodiment 73, wherein said cancer treatment comprises a proteosome inhibitor.
Embodiment 78. The method of Embodiment 73, wherein said cancer treatment comprises an immune checkpoint inhibitor.
Embodiment 79. A method for treating a mammal having a SCD1-associated cancer, wherein said method comprises:

(a) detecting an absence of soft agar colony formation from a cell from a sample obtained from said mammal; and

(b) administering a cancer treatment to said mammal, wherein said cancer treatment is not a SCD1 polypeptide inhibitor.

Embodiment 80. A method for treating a SCD1-associated cancer, wherein said method comprises administering a SCD1 polypeptide inhibitor to a mammal identified as having an absence of soft agar colony formation from a cell from a sample obtained from said mammal.
Embodiment 81. The method of any one of Embodiments 79-80, wherein said mammal is a human.
Embodiment 82. The method of any one of Embodiments 79-81, wherein said sample comprises cancer cells of said cancer.
Embodiment 83. The method of any one of Embodiments 79-82, wherein said cancer is a solid tumor, and wherein said cancer is selected from the group consisting of a liver cancer, a renal cell carcinoma, an ovarian cancer, a breast cancer, a prostate cancer, a colon cancer, a pancreatic cancer, a bladder cancer, a lung cancer, a thyroid cancer, a melanoma, a brain cancer, a stomach cancer, a cervical cancer, a uterine cancer, a chronic lymphocytic leukemia, a, acute lymphocytic leukemia, and a lymphoma.
Embodiment 84. The method of Embodiment 83, wherein said cancer is a liver cancer.
Embodiment 85. The method of Embodiment 84, wherein said liver cancer is a hepatocellular carcinoma.
Embodiment 86. The method of Embodiment 84, wherein said liver cancer is a cholangiocarcinoma.
Embodiment 87. The method of any one of Embodiments 79-86, wherein said cancer treatment is surgery.
Embodiment 88. The method of any one of Embodiments 79-86, wherein said cancer treatment is radiation therapy.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1-17. (canceled)

18. A method for treating a mammal having a SCD1-associated cancer, wherein said method comprises:
(a) detecting a decreased level of p-Src polypeptide expression in a sample obtained from said mammal; and
(b) administering a SCD1 polypeptide inhibitor to said mammal.

19. A method for treating a SCD1-associated cancer, wherein said method comprises administering a SCD1 polypeptide inhibitor to a mammal identified as having a decreased level of p-Src polypeptide expression in a sample obtained from said mammal.

20. The method of claim 18, wherein said mammal is a human.

21. The method of claim 18, wherein said sample comprises cancer cells of said cancer.

22. The method of claim 18, wherein said cancer is a solid tumor, and wherein said cancer is selected from the group consisting of a liver cancer, a renal cell carcinoma, an ovarian cancer, a breast cancer, a prostate cancer, a colon cancer, a pancreatic cancer, a bladder cancer, a lung cancer, a thyroid cancer, a melanoma, a brain cancer, a stomach cancer, a cervical cancer, a uterine cancer, a chronic lymphocytic leukemia, a, acute lymphocytic leukemia, and a lymphoma.

23. The method of claim 22, wherein said cancer is a liver cancer.

24. The method of claim 23, wherein said liver cancer is a hepatocellular carcinoma.

25. The method of claim 23, wherein said liver cancer is a cholangiocarcinoma.

26. The method of claim 18, further comprising detecting an elevated level of c-Myc polypeptide expression in a sample from said mammal.

27. The method of claim 18, further comprising detecting an elevated level of LDHA polypeptide expression in a sample from said mammal.

28-30. (canceled)

31. The method of claim 18, wherein said SCD1 polypeptide inhibitor is a compound having Formula (II) or Formula (IIa): 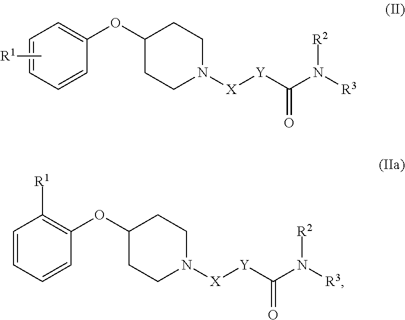
or a pharmaceutically acceptable salt thereof;
wherein:
R¹ is halo;
X is —(C═O)NR⁴—;
Y is 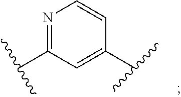 [SMILES: *c1ccnc(*)c1]
and
R², R³, and R⁴ are each independently H or an unsubstituted C₁₋₆alkyl.

32. The method of claim 31, wherein said SCD1 polypeptide inhibitor is SSI-4, 2-{[4-(2-Chlorophenoxy)piperidine-1-carbonyl]amino}-N-methylpyridine-4-carboxamide: 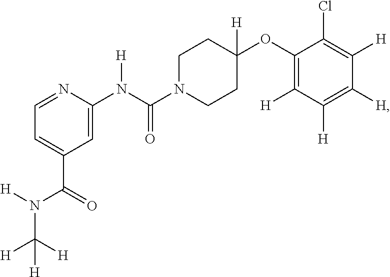
or a pharmaceutically acceptable salt thereof.

33. The method of claim 18, wherein said SCD1 polypeptide inhibitor is a compound having Formula (I) or Formula (Ia): 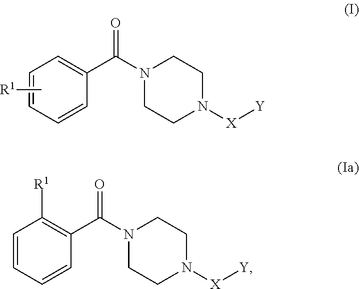
or a pharmaceutically acceptable salt thereof;
wherein:
R¹ is an unsubstituted C₁₋₆alkyl or C₁₋₆haloalkyl;
X is 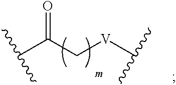
Y is selected from: 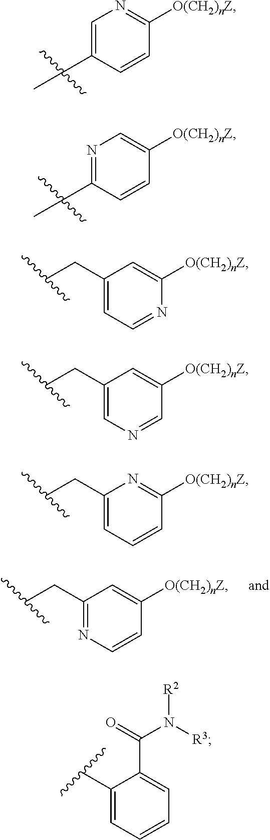
m is 0 or 1;
n is 0, 1, or 2;
V is NR⁴ or O;
R², R³, and R⁴ are each independently H or an unsubstituted C₁₋₆alkyl; and
Z is an unsubstituted aryl.

34. The method of claim 33, wherein said SCD1 polypeptide inhibitor is SSI-2,2-(benzyloxy)-5-{[hydroxy({4-[2-(trifluoromethyl)benzoyl]piperazin-1-yl})methyl]amino}-1,2-dihydropyridin-2-ylium-1-ide: 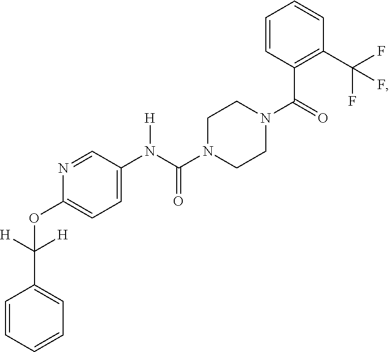 [SMILES: O=C(Nc1ccc(OCc2ccccc2)nc1)N1CCN(C(=O)c2ccccc2C(F)(F)F)CC1]
or a pharmaceutically acceptable salt thereof.

35. The method of claim 18, wherein said method further comprises administering a cancer treatment to said mammal.

36. The method of claim 35, wherein said cancer treatment comprises a kinase inhibitor.

37. The method of claim 36, wherein said kinase inhibitor is regorafenib.

38. The method of claim 35, wherein said cancer treatment comprises a mTOR inhibitor.

39. The method claim 35 wherein said cancer treatment comprises a proteosome inhibitor.

40. The method of claim 35, wherein said cancer treatment comprises an immune checkpoint inhibitor.
